SEQUENCE LISTING (19 sequences)

SEQ ID NO: 1 - protein, 738 aa, Homo sapiens
MRGPSGALWLLLALRTVLGGMEVRWCATSDPEQHKCGNMSEAFREAGIQPSLLCVRGTSADHCVQLIAAQEADAITLDGGAIYEAGKEHGLKPVVGEVYDQEVGTSYYAVAVVRRSSHVTIDTLKGVKSCHTGINRTVGWNVPVGYLVESGRLSVMGCDVLKAVSDYFGGSCVPGAGETSYSESLCRLCRGDSSGEGVCDKSPLERYYDYSGAFRCLAEGAGDVAFVKHSTVLENTDGKTLPSWGQALLSQDFELLCRDGSRADVTEWRQCHLARVPAHAVVVRADTDGGLIFRLLNEGQRLFSHEGSSFQMFSSEAYGQKDLLFKDSTSELVPIATQTYEAWLGHEYLHAMKGLLCDPNRLPPYLRWCVLSTPEIQKCGDMAVAFRRQRLKPEIQCVSAKSPQHCMERIQAEQVDAVTLSGEDIYTAGKTYGLVPAAGEHYAPEDSSNSYYVVAVVRRDSSHAFTLDELRGKRSCHAGFGSPAGWDVPVGALIQRGFIRPKDCDVLTAVSEFFNASCVPVNNPKNYPSSLCALCVGDEQGRNKCVGNSQERYYGYRGAFRCLVENAGDVAFVRHTTVFDNTNGHNSEPWAAELRSEDYELLCPNGARAEVSQFAACNLAQIPPHAVMVRPDTNIFTVYGLLDKAQDLFGDDHNKNGFKMFDSSNYHGQDLLFKDATVRAVPVGEKTTYRGWLGLDYVAALEGMSSQQCSGAAAPAPGAPLLPLLLPALAARLLPPAL

SEQ ID NO: 2 - protein, 11 aa, Homo sapiens
WCATSDPEQHK

SEQ ID NO: 3 - protein, 11 aa, Homo sapiens
RSSHVTIDTLK

SEQ ID NO: 4 - protein, 13 aa, Homo sapiens
SSHVTIDTLKGVK

SEQ ID NO: 5 - protein, 14 aa, Homo sapiens
LCRGDSSGEGVCDK

SEQ ID NO: 6 - protein, 16 aa, Homo sapiens
GDSSGEGVCDKSPLER

SEQ ID NO: 7 - protein, 9 aa, Homo sapiens
YYDYSGAFR

SEQ ID NO: 8 - protein, 7 aa, Homo sapiens
ADVTEWR

SEQ ID NO: 9 - protein, 9 aa, Homo sapiens
VPAHAVVVR

SEQ ID NO: 10 - protein, 10 aa, Homo sapiens
ADTDGGLIFR

SEQ ID NO: 11 - protein, 9 aa, Homo sapiens
CGDMAVAFR

SEQ ID NO: 12 - protein, 11 aa, Homo sapiens
LKPEIQCVSAK

SEQ ID NO: 13 - protein, 12 aa, Homo sapiens
DSSHAFTLDELR

SEQ ID NO: 14 - protein, 13 aa, Homo sapiens
SEDYELLCPNGAR

SEQ ID NO: 15 - protein, 15 aa, Homo sapiens
AQDLFGDDHNKNGFK

SEQ ID NO: 16 - protein, 40 aa, Homo sapiens
FSSEAYGQKDLLFKDSTSELVPIATQTYEAWLGHEYLHAM

SEQ ID NO: 17 - protein, 221 aa, Homo sapiens
ERIQAEQVDAVTLSGEDIYTAGKTYGLVPAAGEHYAPEDSSNSYYVVAVVRRDSSHAFTLDELRGKRSCHAGFGSPAGWDVPVGALIQRGFIRPKDCDVLTAVSEFFNASCVPVNNPKNYPSSLCALCVGDEQGRNKCVGNSQERYYGYRGAFRCLVENAGDVAFVRHTTVFDNTNGHNSEPWAAELRSEDYELLCPNGARAEVSQFAACNLAQIPPHAVM

SEQ ID NO: 18 - protein, 32 aa, Homo sapiens
VRPDTNIFTVYGLLDKAQDLFGDDHNKNGFKM

SEQ ID NO: 19 - protein, 12 aa, Unknown
  Description of Unknown Rattus norvegicus domestica and/or Mus musculus sequence
DSSYSFTLDELR

(19) United States
(12) Patent Application Publication
(10) Pub. No.: US 2021/0355468 A1
(43) Pub. Date: Nov. 18, 2021

(54) COMPOSITIONS AND METHODS FOR TREATING LEWY BODY DEMENTIA

(71) Applicant: BIOASIS TECHNOLOGIES, INC., Burnaby (CA)

(72) Inventors: Deborah Ann Rathjen, Flagstaff Hill (AU); Mei Mei Tian, Maple Ridge (CA)

(21) Appl. No.: 17/320,386

(22) Filed: May 14, 2021

Related U.S. Application Data
(60) Provisional application No. 63/026,187, filed on May 18, 2020.

(51) Int. Cl. C12N 9/24

(52) U.S. Cl. CPC C12N 9/2405; A61K 38/00; C12Y 302/01045; C12Y 306/04006

(57) ABSTRACT

Disclosed are therapeutic payloads comprising p97 fragments coupled with active agents having blood-brain barrier (BBB) transport activity, including variants and combinations thereof, to facilitate delivery of therapeutic or diagnostic agents across the BBB. The therapeutic payloads can be effective in the treatment of Lewy body dementia. Methods of treating Lewy body dementia and pharmaceutical compositions are also disclosed.

### STATEMENT REGARDING THE SEQUENCE LISTINGS

The Sequence Listings associated with this application are provided in text format in lieu of a paper copy and are hereby incorporated by reference into the specification. The name of the text file containing the Sequence Listings is 27942-313-20063-PRO-104457-100-051520.txt. The text file is about 12 KB, was created on May 13, 2021, and is being submitted electronically via EFS-Web.

### TECHNICAL FIELD OF THE INVENTION

The present invention relates to compounds for treating diseases, including compounds that penetrate the blood brain barrier. The invention also provides pharmaceutical compositions comprising compounds of the present invention and methods of using the compositions in the treatment of Lewy Body Dementia (LBD).

### BACKGROUND OF THE INVENTION

Lewy body dementia, which is also known as Lewy body disorder, is a general term used to describe two types of dementia, namely dementia with Lewy Bodies (DLB) and Parkinson's disease dementia (PDD). Both of these dementias are characterized by abnormal protein deposits in the brain, which are known as Lewy bodies. These deposits are named for Dr. Friederich Lewy, a German neurologist, who in 1912 discovered that abnormal protein deposits disrupt the normal functioning of the brain in people with Parkinson's disease. Lewy bodies are composed of the protein alpha-synuclein (α-synuclein). In the healthy brain, alpha-synuclein is involved with the normal functioning of neurons, i.e. nerve cells. However, abnormal Lewy body deposits interfere with neuron function and cause the neurons to die, resulting in the neurological and other symptoms associated with Parkinson's disease dementia and dementia with Lewy bodies. Additionally, Lewy neurites, which are abnormal neuron projections can develop.

Lewy body dementia has been indicated as the second most common neurodegenerative dementia behind Alzheimer's disease, and accounts for about 15 to 30% of all neurodegenerative dementias. See, Velayudhan L, Ffytche D, Ballard C, Aarsland D (September 2017). “New Therapeutic Strategies for Lewy Body Dementias”. Curr Neurol Neurosci Rep(Review). 17 (9): 68. doi:10.1007/s11910-017-0778-2. PMID 28741230, and McKeith I G, Boeve B F, Dickson D W, et al. (July 2017). “Diagnosis and management of dementia with Lewy bodies: Fourth consensus report of the DLB Consortium”. Neurology (Review). 89 (1): 88-100. Also, see, McKeith I G, Boeve B F, Dickson D W, et al. Diagnosis and management of dementia with Lewy bodies: fourth consensus report of the DLB consortium. Neurology. 2017; 89(1): 88-100, and Walker Z, Possin K L, Boeve B F, Aarsland D. Lewy body dementias, Lancet. 2015; 386(10004):1683-1697. Because the protein alpha-synuclein is the major component of Lewy bodies and Lewy neurites, Lewy body dementias are classified as alpha-synucleinopathies. The anatomical distribution of Lewy bodies rather than the severity of the Lewy pathology may be a determining factor of the clinical phenotype of the Lewy body dementia. See, Jellinger K A. Is Braak staging valid for all types of Parkinson's disease? J Neural Transm 2019; 126: 423-431.

Lewy body dementia is a progressive multi-system disease typically involving an array of symptoms associated with cognition, movement, behavior and sleep. Currently available treatments for Lewy body dementias involve medications that are already approved by the Food and Drug Administration to treat symptoms in other diseases, such as Parkinson's disease and Alzheimer's diseases. However, there are no medications available that slow or stop the progression of Lewy body dementia.

The abnormal build-up of alpha-synuclein that is characteristic of Lewy bodies is also correlated with a decrease in the enzyme glucocerebrosidase and abnormal brain levels of the sphingolipid glucocerebroside. Glucocerebrosidase (which is also known as beta-glucosylcerebrosidase, β-glucosylcerebrosidase, or by the abbreviation GCase), is a lysosomal enzyme involved in the breakdown of glucocerebroside (also known as glucosylceramide). Glucocerebroside is needed for normal neuron function. However, this lipid can accumulate abnormally in neuronal tissue when the glucocerebrosidase enzyme is absent or nonfunctional. See, Franco R, Sánchez-Arias J A, Navarro G, Lanciego J L. Glucocerebrosidase Mutations and Synucleinopathies. Potential Role of Sterylglucosides and Relevance of Studying Both GBA1 and GBA2 Genes. Front Neuroanat. 2018; 12:52. Published 2018 Jun. 28. doi:10.3389/fnana.2018.00052.

Post-mortem analysis of brain tissue from patients with Parkinson's disease, and also the lysosomal storage disease, Gaucher disease, has shown correlation between decreases in glucocerebrosidase in the substantia nigra and increases in alpha-synuclein levels. Moreover, Mazzulli et al. has shown that reduced glucosylcerebrosidase activity in cultured neurons resulted in reduced clearance, and subsequently increased levels of alpha-synuclein. See, Gunder A L, Duran-Pacheco G, Zimmermann S, Ruf I, Moors T, Bauman K, et al. Path mediation analysis reveals GBA impacts Lewy body disease status by increasing alpha-synuclein levels. Neurobiol Dis. 2019; 121:205-13; and Mazulli J R, Xu Y-H, Sun Y, Knight A L, McClean P J, Caldwell G A, et al. Gaucher disease glucocerebrosidase and alpha-synuclein form a bidirectional pathogeneic loop in synucleinopathies. Cell. 2011; 146(1):37-52. It has been suggested that under lysosomal conditions, glucocerebrosidase can prevent the aggregation of alpha-synuclein, thereby providing a molecular explanation for its connection to Parkinson's disease.

The mechanistic pathways involved with brain tissue metabolism are yet even more complicated. The biosynthesis of glucocerebroside, is mediated by the enzyme glucosylceramide synthase. Therefore, the inhibition of glucosylceramide synthase could provide a further means to control abnormal levels of glucocerebroside in brain tissue.

In addition to the biochemical mechanistic pathways involved in maintaining brain tissue health, there are also genetic considerations. The GBA-1 gene encodes for glucocerebrosidase. Mutations of this gene is known for causing Gaucher disease, the most common autosomal recessive lysosomal storage disease, where homozygous mutations in this gene leads to systemic and neurological manifestations of varying severities caused by lysosomal build-up of the substrate glucocerebroside and reduced clearance. To date, more than 300 mutations have been identified with mild to severe biological consequences and clinical presentations (age of onset or progression rate). See, Schneider S A, Alcalay R N. Precision medicine in Parkinson's disease: emerging treatments for genetic Parkinson's disease. J Neurol. 2020; 267(3):860-869. doi:10.1007/s00415-020-09705-7. GBA-1 mutations have also been shown to have a strong link to synucleinopathies. GBA-1 mutations were found to be the most common genetic risk factor for developing Parkinson's disease with some mutations estimated to potentially cause a 20- to 30-fold increase in risk. See, Sidransky, E., Nails, M. A., Aasly, J. O., Aharon-Peretz, J., Annesi, G., Barbosa, E. R., et al. (2009). Multicenter analysis of glucocerebrosidase mutations in Parkinson's disease. N. Engl. J. Med. 361, 1651-1661.doi: 10.1056/NEJMoa0901281; Standaert, D. G. (2017). What would Dr. James Parkinson think today? Mutations in β-glucocerebrosidase and risk of Parkinson's disease. Mov. Disord. 32, 1341-1342. doi: 10.1002/mds.27206; O'Regan, G., deSouza, R. M., Balestrino, R., and Schapira, A. H. (2017). Glucocerebrosidase mutations in Parkinson disease. J. Parkinsons Dis. 7,411-422. doi: 10.3233/JPD-171092; Migdalska-Richards, A., and Schapira, A. H. V. (2016). The relationship between glucocerebrosidase mutations and Parkinson disease. J. Neurochem. 139, 77-90. doi: 10.1111/jnc.13385; and Franco R, Sánchez-Arias J A, Navarro G, Lanciego J L. Glucocerebrosidase Mutations and Synucleinopathies. Potential Role of Sterylglucosides and Relevance of Studying Both GBA1 and GBA2 Genes. Front Neuroanat. 2018; 12:52. Published 2018 Jun. 28. doi:10.3389/fnana.2018.00052.

Additionally, genetic mutations have been associated with Lewy body dementias. See, Mata, I. F., Samii, A., Schneer, S. H., Roberts, J. W., Griffith, A., Leis, B. C., et al. (2008). Glucocerebrosidase gene mutations. Arch. Neurol. 65, 379-382, and Nails, M. A., Duran, R., Lopez, G., Kurzawa-Akanbi, M., McKeith, I. G., Chinnery, P. F., et al. (2013). A multicenter study of glucocerebrosidase mutations in dementia with Lewy bodies. JAMA Neurol. 70, 727-735.doi: 10.1001/jamaneurol.2013.1925. doi:

As seen from the foregoing, a complicated group of biochemical pathways and genetic mechanisms are involved with brain tissue health and function and the development of disease states.

Overcoming the difficulties of delivering therapeutic agents to specific regions of the brain represents a major challenge for the treatment or diagnosis of many central nervous system (CNS) disorders, including those of the brain such as Lewy body dementia and Parkinson's disease. In its neuroprotective role, the blood-brain barrier (BBB) functions to hinder the beneficial delivery of many potentially important therapeutic agents to the brain.

Therapeutic agents that might otherwise be effective in diagnosis and therapy do not cross the blood-brain barrier in adequate amounts. It is reported that over 95% of all therapeutic molecules do not cross the blood-brain barrier. Accordingly, it is desired to deliver therapeutic agents across the blood-brain barrier to treat diseases.

Enzyme replacement therapy (ERT) is available for patients with neurological disorders such as the group of Gaucher diseases. This enzyme therapy focuses on the restoration of glucocerbrosidase activity. Enzyme replacement therapy typically involves patients receiving intravenous (IV) infusions about every 2 weeks, either at an infusion center or at home.

The United States Food and Drug Administration (FDA) has approved enzyme replacement therapy treatments for Gaucher disease including the following enzyme replacement therapy drugs: Cerezyme™ (imiglucerase), available from Genzyme Corporation, Cambridge, Mass.; VPRIV™ (velaglucerase alfa), available from Shire Human Genetic Therapies, Inc., Lexington, Mass.; and Elelyso™ (taliglucerase alfa), available from Pfizer Laboratories, New York, N.Y. However, enzymes are high molecular weight materials which are difficult to synthesize or isolate. For example, glucocerebrosidase is a protein having 497 amino acids.

Another approach to treatment is with small molecule drugs, i.e. compounds having a molecular weight of about 1000 or less. Substrate reduction therapy (SRT) utilizes oral medications that decrease the amount of glucocerebroside that the body makes, reducing excess buildup. Substrate reduction therapy blocks the body from producing glucocerebroside, the fatty chemical that builds up in the bodies of patients with Gaucher disease, by inhibiting glucosylceramide synthase, which is involved with the biosynthesis of glucocerebroside. There are currently two FDA-approved oral substrate reduction therapy drugs for treating patients with Gaucher disease: Cerdelga™ (eliglustat), available from Genzyme Corporation, Cambridge, Mass.; and Zavesca™ (miglustat), available from Actelion Pharmaceuticals US Inc., South San Francisco, Calif. Even though these are lower molecular weight materials, their delivery is still a challenge.

The fundamental issue for both enzyme replacement therapy and substrate reduction therapy is that neither of these means are effective for delivering drugs, whether an enzyme or a small molecule, across the blood-brain barrier. Therefore, it is difficult to intervene directly in the brain to effect the neurological symptoms present in Gaucher disease. Furthermore, the treatment of Lewy body dementia is even farther behind.

Regarding Lewy body dementia, there is currently an unmet need for delivering therapeutic and diagnostic agents across the blood brain barrier and into the brain for degrading or preventing the formation of Lewy bodies, and for degrading or preventing the formation of unwanted brain levels of glucocerebroside and alpha-synuclein, and thereby treating or preventing the resulting dementias. There is a need for the development of technologies for targeting the different biochemical pathways involved in normal brain metabolism and function. These technologies can include the delivery of enzymatic agents such as glucocerebrosidase, and for delivering inhibitors, such as glucosylceramide synthase inhibitors. The present invention provides novel compositions and methods for delivering active agents across the blood brain barrier to treat, prevent, or slow the progression of Lewy body dementias.

### SUMMARY OF THE INVENTION

In accordance with the present invention, there are provided methods of treating Lewy body dementias (Dementia of Lewy bodies and Parkinson's disease dementia) in a subject by administering a therapeutic payload that comprises an active agent suitable for treating Lewy body dementias coupled with a certain p97 fragment which enables the active agent to cross the BBB. In accordance with the present invention, the therapeutic payload has pharmacokinetic properties that are similar to the active agent in a form that is uncoupled to the p97 fragment.

By virtue of the present invention, it may now be possible to treat Lewy body dementias by promoting the transport of the active agent across the blood brain barrier of the subject.

In another aspect of the present invention provides the following embodiments:

1. A method of treating Lewy body dementia comprising administering to a subject in need thereof a therapeutic payload (i.e. composition) comprising an active agent suitable for treating Lewy body dementia coupled with a p97 polypeptide or fragment thereof, wherein said administration promotes the transport of the therapeutic payload across the blood brain barrier of the subject.
2. A method according to embodiment 1 wherein said subject is a mammal.
3. A method according to embodiment 2 wherein said mammal is a human.
4. A method according to any of embodiments 1 to 3 wherein the p97 polypeptide comprises up to about 300 amino acids in length, where the polypeptide comprises an amino acid sequence at least 70% identical to DSSHAFTLDELR (SEQ ID NO:13) or any one or more of SEQ ID NOS: 2 to 19.
5. A method according to any of embodiments 1 to 3 wherein the p97 polypeptide comprises DSSHAFTLDELR (SEQ ID NO:13) or any one or more of SEQ ID NOS: 2 to 19, optionally including adjacent C-terminal and/or N-terminal sequences as defined by SEQ ID NO:1.
6. A method according to any of embodiments 1 to 3 wherein the p97 polypeptide comprises 2, 3, 4, or 5 amino acids of DSSHAFTLDELR (SEQ ID NO:13) or SEQ ID NOS: 2 to 19, optionally including any intervening sequences as defined by SEQ ID NO:1.
7. A method according to any of embodiments 1 to 3 wherein the p97 polypeptide comprises one or both of SEQ ID NO:13 and/or 14, optionally including intervening sequences as defined by SEQ ID NO:1.
8. A method according to any of embodiments 1 to 7 wherein the p97 polypeptide comprises up to about 250, 200, 150, 100, 50, 20, or 10 amino acids in length.
9. A method according to any of embodiments 1 to 8 wherein said active agent is coupled to said p97 polypeptide or fragment thereof with a linker.
10. A method according to any of embodiments 1-3 or 9 wherein said polypeptide or fragment thereof comprises a peptide corresponding to SEQ ID NO: 13 [DSSHAFTLDELR] or a sequence having at least about 70% or more homology thereto, or having at least about 75% or more homology thereto, or having at least about 80% or more homology thereto, or having at least about 85% or more homology thereto, or having at least about 90% or more homology thereto, or having at least about 95% or more homology thereto, or having at least about 99% or more homology thereto.
11. A method according to embodiment 1 wherein said active agent is a lysosomal enzyme.
12. A method according to embodiment 1 wherein said active agent is capable of degrading, cleaving, hydrolyzing, inhibiting, or preventing the formation of glucocerebroside.
13. A method according to embodiment 11 wherein said active agent is capable of degrading, cleaving, hydrolyzing, inhibiting, or preventing the formation of glucocerebroside in the brain tissue of the subject.
14. A method according to any of embodiments 11 to 13 wherein said active agent has glucosylceramidase activity.
15. A method according to any of embodiments 11 to 14 wherein said active agent is Beta-glucocerebrosidase (β-glucocerebrosidase; GCase) or a derivative or analogue thereof.
16. A method according to embodiment 15 wherein said active agent is human Beta-glucocerebrosidase or a derivative or analogue thereof.
17. A method according to embodiment 15 wherein said active agent is a recombinant Beta-glucocerebrosidase (an active form thereof) or a derivative or analogue thereof.
18. A method according to embodiment 16 or 17 wherein said active agent is produced by gene activation technology in a human fibroblast cell line.
19. A method according to embodiment 16 or 17 wherein said active agent is produced by gene activation technology in a Chinese Hamster Ovary (CHO) cell line.
20. A method according to any of embodiments 15 to 19 wherein said active agent is selected from the group consisting of alglucerase (from human placental tissue and also previously known by the tradename Ceredase), imiglucerase (also known by the tradename Cerezyme), velaglucerase (or velalgucerase alpha) (also known by the tradenanme Vpriv), or taliglucerase (or taliglucerase alfa) (also known by the tradename Elelyso), and combinations thereof.
21. A method according to embodiment 20 wherein said active agent is alglucerase (from human placental tissue and also previously known by the tradename Ceredase).
22. A method according to embodiment 20 wherein said active agent is imiglucerase (also known by the tradename Cerezyme).
23. A method according to embodiment 20 wherein said active agent is velaglucerase (or velalgucerase alpha) (also known by the tradenanme Vpriv).
24. A method according to embodiment 20 wherein said active agent is taliglucerase (or taliglucerase alfa) (also known by the tradename Elelyso).
25. A method according to embodiment 1 wherein said active agent is a small molecule drug (i.e. a drug molecular generally having a molecular weight less than about 1000 grams/mole, or less than about 750 grams/mole, or less thana about 500 grams/mole.
26. A method according to embodiment 1 wherein said Lewy body dementia is selected from dementia with Lewy bodies and Parkinson's disease dementia.
27. A method according to embodiment 26 wherein said Lewy body dementia is dementia with Lewy bodies.
28. A method according to embodiment 26 wherein said Lewy body dementia is Parkinson's disease dementia.
29. A method according to embodiment 1 wherein said Lewy body dementia is associated with a deficiency or absence of Beta-glucocerebrosidase.
30. A method according to embodiment 1 wherein said Lewy body dementia is associated with a deficiency or absence of Beta-glucocerebrosidase in lysosomes.
31. A method according to embodiment 30 wherein said Lewy body dementia is associated with a deficiency or absence of Beta-glucocerebrosidase in lysosomes in the brain.
32. A method according to embodiment 1 for degrading Lewy bodies in the brain.
33. A method according to embodiment 1 for decreasing the number of Lewy bodies in the brain.
34. A method according to embodiment 1 for preventing or slowing the formation or accumulation of Lewy bodies in the brain.
35. A method according to embodiment 1 for degrading, decreasing, preventing or slowing the formation or accumulation of abnormal protein deposits in the brain.
36. A method according to any of embodiments 32 to 35 wherein said Lewy bodies or protein deposits are comprised of alpha-synuclein.
37. A method according to embodiment 36 wherein said Lewy bodies or protein deposits further comprise a glycosphingolipid.
38. A method according to embodiment 37 wherein said glycosphingolipid is glucocerebroside.
39. A method according to embodiment 1 wherein said therapeutic payload (i.e. composition) is administered to a regimen selected from the group consisting of at least about once per day, or at least about every other day, or at least aboue two times per week, or at least about 1 time per week, or at least about 1 time every two weeks, or at least about 1 time per month (or about 1 time every 30 days).
40. A method according to any of embodiments 1 to 39 wherein said subject exhibits an improvement in at least one clinical marker, evaluation, or assessment for Lewy body dementia, and optionally wherein said improve is at least about 5%, or is at least about 10%, or is at least about 15%, or is at least about 20%, or is at least about 25%, compared to baseline before the application of the method.
41. A method according to embodiment 40 wherein said clinical marker, evaluation, or assessment is made using the Lewy Body Composite Risk Score (LBCRS) (which can be further defined as a one-page survey with structured yes/no questions for six non-motor features that are present in subjects with Lewy body dementia, but are much less commonly found in other forms of dementia).
42. A composition for use in the manufacture of a medicament for treating Lewy body dementia comprising administering to a subject in need thereof a therapeutic payload comprising an active agent suitable for treating Lewy body dementia coupled with a p97 polypeptide or fragment thereof, wherein said administration promotes the transport of the therapeutic payload across the blood brain barrier of the subject.
43. A conjugate comprising p97 or a fragment thereof that is conjugated to an active agent suitable for treating Lewy body dementia to form aconjugate-p97-actrve agent conjugate wherein the p97 fragment comprises, consists essentially of, or consists of DSSHAFTLDELR (SEQ ID NO: 13), or a sequence having at least about 70% or more homology thereto, or having at least about 75% or more homology thereto, or having at least about 80% or more homology thereto, or having at least about 85% or more homology thereto, or having at least about 90% or more homology thereto, or having at least about 95% or more homology thereto, or having at least about 99% or more homology thereto.
44. A conjugate according to embodiment 43 wherein the p97 fragment has one or more terminal cysteines and/or tyrosines.
45. A conjugate according to embodiment 43 or 44 wherein the p97 fragment comprises, consists essentially of, or consists of DSSHAFTLDELR (SEQ ID NO: 13) with a C-terminal tyrosine, and wherein the p97 fragment and the active agent are separated by a peptide linker of about 1-20 amino acids in length.
46. A conjugate according to embodiment 43 or 44 wherein the p97 fragment comprises, consists essentially of, or consists of DSSHAFTLDELR (SEQ ID NO: 13) with a C-terminal cysteine, and wherein the p97 fragment and the active agent are separated by a peptide linker of about 1-20 amino acids in length.
47. A conjugate according to embodiment 43 or 44 wherein the p97 fragment comprises, consists essentially of, or consists of DSSHAFTLDELR (SEQ ID NO: 13) with a N-terminal tyrosine, and wherein the p97 fragment and the active agent are separated by a peptide linker of about 1-20 amino acids in length.
48. A conjugate according to embodiment 43 or 44 wherein the p97 fragment comprises, consists essentially of, or consists of DSSHAFTLDELR (SEQ ID NO: 13) with a N-terminal cysteine, and wherein the p97 fragment and the active agent are separated by a peptide linker of about 1-20 amino acids in length.
49. A conjugate according to embodiment 43 or 44 wherein the p97 fragment comprises, consists essentially of, or consists of DSSHAFTLDELR (SEQ ID NO: 13) with a C-terminal tyrosine cysteine dipeptide, and wherein the p97 fragment and the active agent are separated by a peptide linker of about 1-20 amino acids in length.
50. A conjugate according to embodiment 43 or 44 wherein the p97 fragment comprises, consists essentially of, or consists of DSSHAFTLDELR (SEQ ID NO: 13) with a N-terminal tyrosine cysteine dipeptide, and wherein the p97 fragment and the active agent are separated by a peptide linker of about 1-20 amino acids in length.
51. A conjugate comprising p97 or a fragment thereof that is conjugated to an active agent suitable for treating Lewy body dementia to form aconjugate-p97-actrve agent conjugate wherein the p97 fragment comprises, consists essentially of, or consists of DSSYSFTLDELR (SEQ ID NO: 19), or a sequence having at least about 70% or more homology thereto, or having at least about 75% or more homology thereto, or having at least about 80% or more homology thereto, or having at least about 85% or more homology thereto, or having at least about 90% or more homology thereto, or having at least about 95% or more homology thereto, or having at least about 99% or more homology thereto.
52. A conjugate according to embodiment 51 wherein the p97 fragment has one or more terminal cysteines and/or tyrosines.
53. A conjugate according to embodiment 51 or 52 wherein the p97 fragment comprises, consists essentially of, or consists of DSSYSFTLDELR (SEQ ID NO: 19) with a C-terminal tyrosine, and wherein the p97 fragment and the active agent are separated by a peptide linker of about 1-20 amino acids in length.
54. A conjugate according to embodiment 51 or 52 wherein the p97 fragment comprises, consists essentially of, or consists of DSSYSFTLDELR (SEQ ID NO: 19) with a C-terminal cysteine, and wherein the p97 fragment and the active agent are separated by a peptide linker of about 1-20 amino acids in length.
55. A conjugate according to embodiment 51 or 52 wherein the p97 fragment comprises, consists essentially of, or consists of DSSYSFTLDELR (SEQ ID NO: 19) with a N-terminal tyrosine, and wherein the p97 fragment and the active agent are separated by a peptide linker of about 1-20 amino acids in length.
56. A conjugate according to embodiment 51 or 52 wherein the p97 fragment comprises, consists essentially of, or consists of DSSYSFTLDELR (SEQ ID NO: 19) with a N-terminal cysteine, and wherein the p97 fragment and the active agent are separated by a peptide linker of about 1-20 amino acids in length.
57. A conjugate according to embodiment 51 or 52 wherein the p97 fragment comprises, consists essentially of, or consists of DSSYSFTLDELR (SEQ ID NO: 19) with a C-terminal tyrosine cysteine dipeptide, and wherein the p97 fragment and the active agent are separated by a peptide linker of about 1-20 amino acids in length.
58. A conjugate according to embodiment 51 or 52 wherein the p97 fragment comprises, consists essentially of, or consists of DSSYSFTLDELR (SEQ ID NO: 19) with a N-terminal tyrosine cysteine dipeptide, and wherein the p97 fragment and the active agent are separated by a peptide linker of about 1-20 amino acids in length.
59. A conjugate according to any of embodiments 43 to 58 wherein said active agent is a lysosomal enzyme.
60. A conjugate according to embodiment 59 wherein said active agent is capable of degrading, cleaving, hydrolyzing, inhibiting, or preventing the formation of glucocerebroside.
61. A conjugate according to embodiment 59 wherein said active agent is capable of degrading, cleaving, hydrolyzing, inhibiting, or preventing the formation of glucocerebroside in the brain tissue of the subject.
62. A conjugate according to any of embodiments 59 to 61 wherein said active agent has glucosylceramidase activity.
63. A conjugate according to any of embodiments 59 to 62 wherein said active agent is Beta-glucocerebrosidase (β-glucocerebrosidase; GCase) or a derivative or analogue thereof.
64. A conjugate according to embodiment 63 wherein said active agent is human Beta-glucocerebrosidase or a derivative or analogue thereof.
65. A conjugate according to embodiment 63 wherein said active agent is a recombinant Beta-glucocerebrosidase (an active form thereof) or a derivative or analogue thereof.
66. A conjugate according to embodiment 64 or 65 wherein said active agent is produced by gene activation technology in a human fibroblast cell line.
67. A conjugate according to embodiment 64 or 65 wherein said active agent is produced by gene activation technology in a Chinese Hamster Ovary (CHO) cell line.
68. A conjugate according to any of embodiments 62 to 66 wherein said active agent is selected from the group consisting of alglucerase (from human placental tissue and also previously known by the tradename Ceredase), imiglucerase (also known by the tradename Cerezyme), velaglucerase (or velalgucerase alpha) (also known by the tradenanme Vpriv), or taliglucerase (or taliglucerase alfa) (also known by the tradename Elelyso), and combinations thereof.
69. A conjugate according to embodiment 68 wherein said active agent is alglucerase (from human placental tissue and also previously known by the tradename Ceredase).
70. A conjugate according to embodiment 68 wherein said active agent is imiglucerase (also known by the tradename Cerezyme).
71. A conjugate according to embodiment 68 wherein said active agent is velaglucerase (or velalgucerase alpha) (also known by the tradenanme Vpriv).
72. A conjugate according to embodiment 68 wherein said active agent is taliglucerase (or taliglucerase alfa) (also known by the tradename Elelyso).
73. A conjugate according to any of embodiments 43 to 58 wherein said active agent is a small molecule drug (i.e. a drug molecular generally having a molecular weight less than about 1000 grams/mole, or less than about 750 grams/mole, or less thana about 500 grams/mole.

These and other aspects of the present invention will become apparent from the disclosure herein.

### DETAILED DESCRIPTION OF CERTAIN EMBODIMENTS

The following detailed description is provided to aid those skilled in the art in practicing the present invention. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

The term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

The term “administering” refers to the physical introduction of a composition comprising a therapeutic agent to a subject, using any of the various methods and delivery systems known to those skilled in the art. For example, routes of administration can include bucal, intranasal, ophthalmic, oral, osmotic, parenteral, rectal, sublingual, topical, transdermal, vaginal intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Administering can also be performed, for example, once, a plurality of times, and/or over one or more extended periods and can be a therapeutically effective dose or a subtherapeutic dose.

As used herein, the term “amino acid” is intended to mean both naturally occurring and non-naturally occurring amino acids as well as amino acid analogs and mimetics. Naturally occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics Arginine (Arg or R) would have a positive charge moiety located in similar molecular space and having the same degree of mobility as thee-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “conjugate” is intended to refer to the entity formed as a result of covalent or non-covalent attachment or linkage of an agent or other molecule, e.g., a biologically active molecule, to a p97 polypeptide. One example of a conjugate polypeptide is a “fusion protein” or “fusion polypeptide,” that is, a polypeptide that is created through the joining of two or more coding sequences, which originally coded for separate polypeptides; translation of the joined coding sequences results in a single, fusion polypeptide, typically with functional properties derived from each of the separate polypeptides.

As used herein, the terms “function” and “functional” and the like refer to a biological, enzymatic, or therapeutic function.

“Homology” refers to the percentage number of amino acids that are identical or constitute conservative substitutions. Homology may be determined using sequence comparison programs such as GAP (Deveraux et al., Nucleic Acids Research. 12, 387-395, 1984), which is incorporated herein by reference. In this way sequences of a similar or substantially different length to those cited herein could be compared by insertion of gaps into the alignment, such gaps being determined, for example, by the comparison algorithm used by GAP.

By “isolated” is meant material that is substantially or essentially free from components that normally accompany it in its native state. For example, an “isolated peptide” or an “isolated polypeptide” and the like, as used herein, includes the in vitro isolation and/or purification of a peptide or polypeptide molecule from its natural cellular environment, and from association with other components of the cell; i.e., it is not significantly associated with in vivo substances.

The term “linkage,” “linker,” “linker moiety,” or “L” is used herein to refer to a linker that can be used to separate a p97 polypeptide fragment from an agent of interest, or to separate a first agent from another agent, for instance where two or more agents are linked to form a p97 conjugate. The linker may be physiologically stable or may include a releasable linker such as an enzymatically degradable linker (e.g., proteolytically cleavable linkers). In certain aspects, the linker may be a peptide linker, for instance, as part of a p97 fusion protein. In some aspects, the linker may be a non-peptide linker or non-proteinaceous linker. In some aspects, the linker may be particle, such as a nanoparticle.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the amount produced by no composition (e.g., the absence of polypeptide of conjugate of the invention) or a control composition, sample or test subject. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount produced by no composition or a control composition, including all integers in between. As one non-limiting example, a control could compare the activity, such as the amount or rate of transport/delivery across the blood brain barrier, the rate and/or levels of distribution to central nervous system tissue, and/or the Cmax for plasma, central nervous system tissues, or any other systemic or peripheral non-central nervous system tissues, of a p97-agent conjugate relative to the agent alone. Other examples of comparisons and “statistically significant” amounts are described herein.

In certain embodiments, the “purity” of any given agent (e.g., a p97 conjugate such as a fusion protein) in a composition may be specifically defined. For instance, certain compositions may comprise an agent that is at least 80%, 85%, 90%, 91%, 92%, 93%, 94%, 95%, 96%, 97%, 98%, 99%, or 100% pure, including all decimals in between, as measured, for example and by no means limiting, by high pressure liquid chromatography (HPLC), a well-known form of column chromatography used frequently in biochemistry and analytical chemistry to separate, identify, and quantify compounds.

The terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. The polypeptides described herein are not limited to a specific length of the product; thus, peptides, oligopeptides, and proteins are included within the definition of polypeptide, and such terms may be used interchangeably herein unless specifically indicated otherwise. The polypeptides described herein may also comprise post-expression modifications, such as glycosylations, acetylations, phosphorylations and the like, as well as other modifications known in the art, both naturally occurring and non-naturally occurring. A polypeptide may be an entire protein, or a subsequence, fragment, variant, or derivative thereof.

A “physiologically cleavable” or “hydrolyzable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include, but are not limited to: carboxylate ester, phosphate ester, anhydride, acetal, ketal, acyloxyalkyl ether, imine, orthoester, thio ester, thiol ester, carbonate, and hydrazone, peptides and oligonucleotides.

A “releasable linker” includes, but is not limited to, a physiologically cleavable linker and an enzymatically degradable linker. Thus, a “releasable linker” is a linker that may undergo either spontaneous hydrolysis, or cleavage by some other mechanism (e.g., enzyme-catalyzed, acid-catalyzed, base-catalyzed, and so forth) under physiological conditions. For example, a “releasable linker” can involve an elimination reaction that has a base abstraction of a proton, (e.g., an ionizable hydrogen atom, Ha), as the driving force. For purposes herein, a “releasable linker” is synonymous with a “degradable linker.” An “enzymatically degradable linkage” includes a linkage, e.g., amino acid sequence that is subject to degradation by one or more enzymes, e.g., peptidases or proteases. In particular embodiments, a releasable linker has a half life at pH 7.4, 25° C., e.g., a physiological pH, human body temperature (e.g., in vivo), of about 30 minutes, about 1 hour, about 2 hour, about 3 hours, about 4 hours, about 5 hours, about 6 hours, about 12 hours, about 18 hours, about 24 hours, about 36 hours, about 48 hours, about 72 hours, or about 96 hours or less.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, lie, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing), typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity.” A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997. A detailed discussion of sequence analysis can be found in Unit 19.3 of Ausubel et al., “Current Protocols in Molecular Biology,” John Wiley & Sons Inc, 1994-1998, Chapter 15.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

The term “solubility” refers to the property of a p97 polypeptide fragment or conjugate to dissolve in a liquid solvent and form a homogeneous solution. Solubility is typically expressed as a concentration, either by mass of solute per unit volume of solvent (g of solute per kg of solvent, g per dL (100 ml), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, or pH 7.4. In certain embodiments, solubility is measured in water or a physiological buffer such as PBS or NaCl (with or without NaP). In specific embodiments, solubility is measured at relatively lower pH (e.g., pH 6.0) and relatively higher salt (e.g., 500 mM NaCl and 10 mM NaP). In certain embodiments, solubility is measured in a biological fluid (solvent) such as blood or serum. In certain embodiments, the temperature can be about room temperature (e.g., about 20, 21, 22, 23, 24, 25° ( ) or about body temperature (−37° C.). In certain embodiments, a p97 polypeptide or conjugate has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, or 30 mg/ml at room temperature or at about 37° C.

A “subject,” as used herein, includes any animal that exhibits a symptom, or is at risk for exhibiting a symptom, which can be treated or diagnosed with a p97 conjugate of the invention. Suitable subjects (patients) include laboratory animals (such as mouse, rat, rabbit, or guinea pig), farm animals, and domestic animals or pets (such as a cat or dog). Non-human primates and, preferably, human patients, are included.

“Substantially” or “essentially” means nearly totally or completely, for instance, 95%, 96%, 97%, 98%, 99% or greater of some given quantity.

“Substantially free” refers to the nearly complete or complete absence of a given quantity for instance, less than about 10%, 5%, 4%, 3%, 2%, 1%, 0.5% or less of some given quantity. For example, certain compositions may be “substantially free” of cell proteins, membranes, nucleic acids, endotoxins, or other contaminants.

“Treatment” or “treating,” as used herein, includes any desirable effect on the symptoms or pathology of a disease or condition, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art.

The term “wild-type” refers to a gene or gene product that has the characteristics of that gene or gene product when isolated from a naturally-occurring source. A wild type gene or gene product (e.g., a polypeptide) is that which is most frequently observed in a population and is thus arbitrarily designed the “normal” or “wild-type” form of the gene.

Lewy Bodies and Lewy Body Dementia

As described above, Lewy body dementia, also known as Lewy body disorder, is a general term used to describe two types of dementia, namely dementia with Lewy Bodies and Parkinson's disease dementia. The disorders are characterized by abnormal protein aggregates or deposits in the brain known as Lewy bodies. Lewy bodies are composed of the protein alpha-synuclein. The condition is also associated with low levels or activity of glucocerebrosidase and abnormal increases in glucoderebroside. Both alph-synuclein and glucocerbroside are needed for normal functioning of brain neurons. However, the abnormal Lewy body deposits interfere with neuron function and cause neuron death resulting in the neurological and other symptoms associated with Parkinson's disease (PD) and Lewy Body Dementia.

Lewy bodies are microscopically identified when histology is performed on the brain at the time of autopsy, thereby confirming diagnosis of a suspected disease sufferer. The Lewy bodies can be found in the brainstem, such as within the substantia nigra, or within the cortex Lewy bodies are generally spherical in shape and displace other cell components. The Lewy body is an eosinophilic cytoplasmic inclusion having of a dense core. This core is surrounded by a halo (approximate 10-nm in width), with the halo having radiating fibrils.

As the disease progresses, Lewy bodies form deposits in the neurons and typically manifest in the areas of the brain that control memor and movement. Also, the biochemical function of brain chemicals can be affected. The Lewy bodies therefore cause damage to the brain because of interference with neuron function and by causing cell death.

p97 Polypeptide Sequences and Conjugates Thereof

The compositions and methods of the present invention comprise conjugates of p97 or fragments thereof. The p97 protein and fragments useful herein are described in PCT Patent Application Publication No. WO 2003/057179, to Starr et al., published Jul. 3, 2003; U.S. Pat. No. 8,546,319, to Starr et al., issued Mar. 23, 2010; PCT Patent Application Publication No. WO 2014/160438, to Vitalis et al., published Oct. 2, 2014; U.S. Pat. No. 9,364,567, to Vitalis et al., issued Jun. 14, 2016; U.S. Pat. No. 9,993,530, to Vitalis et al., issued May 12, 2016; and PCT Patent Application Publication No. WO 2019/231725, to Tian et al., published Dec. 5, 2019; which are all incorporated by reference herein in their entirety.

Embodiments of the present invention relate generally to polypeptide fragments of p97, and particularly of human p97 (melanotransferrin; MTf, SEQ ID NO: 1), compositions that comprise such fragments, and conjugates thereof. In certain instances, the p97 polypeptide fragments described herein have transport activity, that is, they are ability to transport across the blood-brain barrier (BBB). In particular embodiments, the p97 fragments are covalently, non-covalently, or operatively coupled to an agent of interest, such as a therapeutic, diagnostic, or detectable agent, to form a p97-agent conjugate. Specific examples of agents include small molecules and polypeptides, such as antibodies, among other agents described herein and known in the art. Exemplary p97 polypeptide sequences and agents are described below. Also described are exemplary methods and components, such as linker groups, for coupling a p97 polypeptide to an agent of interest. p97 Sequence. In some embodiments, a p97 polypeptide comprises, consists essentially of, or consists of the human p97 fragments identified in SEQ ID NO: 13 (DSSHAFTLDELR).

In other specific embodiments, described in greater detail below, a p97 polypeptide sequence comprises a sequence having at least 70%, 75%, 80%, 85%, 90%, 95%, 96%, 97%, 98%, or 99% identity or homology, along its length, to the human p97 sequence set forth in SEQ ID NO: 13.

In particular embodiments, the p97 fragment or variant thereof has the ability to cross the BBB, and optionally transport an agent of interest across the BBB and into the central nervous system. In certain embodiments, the p97 fragment or variant thereof is capable of specifically binding to a p97 receptor, an LRPI receptor, and/or an LRPIB receptor.

In some embodiments, the p97 fragment has one or more terminal (e.g., N-terminal, C-terminal) cysteines and/or tyrosines, which can be added for conjugation and iodination, respectively. In some embodiments, a tyrosine cysteine dipeptide can by used.

In certain aspects of the invention, including for example some or all of the aspects described herein, the p97 fragment DSSHAFTLDELR (SEQ ID NO: 13) can be replaced with the p97 fragment DSSYSFTLDELR (SEQ ID NO: 19).

Table 1 provides SEQ ID NOS: 1 to 19 for the full length human p97 (SEQ ID NO: 1) and fragments thereof (SEQ ID NOS: 2-18), as well as an exemplary rat/mouse fragment (SEQ ID NO: 19), useful in the present invention.

TABLE 1

| SEQ ID NO: and | |
|---|---|
| Species | Amino AcidSequence |
| SEQ ID NO: 1 | Met Arg Gly Pro Ser Gly Ala Leu Trp Leu Leu Leu Ala Leu Arg Thr |
| Homo Sapiens | Val Leu Gly Gly Met Glu Val Arg Trp Cys Ala Thr Ser Asp Pro Glu |
| | Gln His Lys Cys Gly Asn Met Ser Glu Ala Phe Arg Glu Ala Gly Ile |
| | Gln Pro Ser Leu Leu Cys Val Arg Gly Thr Ser Ala Asp His Cys Val |
| | Gln Leu Ile Ala Ala Gln Glu Ala Asp Ala Ile Thr Leu Asp Gly Gly Ala |
| | Ile Tyr Glu Ala Gly Lys Glu His Gly Leu Lys Pro Val Val Gly Glu Val |
| | Tyr Asp Gln Glu Val Gly Thr Ser Tyr Tyr Ala Val Ala Val Val Arg Arg |
| | Ser Ser His Val Thr Ile Asp Thr Leu Lys Gly Val Lys Ser Cys His |
| | Thr Gly Ile Asn Arg Thr Val Gly Trp Asn Val Pro Val Gly Tyr Leu Val |
| | Glu Ser Gly Arg Leu Ser Val Met Gly Cys Asp Val Leu Lys Ala Val |
| | Ser Asp Tyr Phe Gly Gly Ser Cys Val Pro Gly Ala Gly Glu Thr Ser |
| | Tyr Ser Glu Ser Leu Cys Arg Leu Cys Arg Gly Asp Ser Ser Gly Glu |
| | Gly Val Cys Asp Lys Ser Pro Leu Glu Arg Tyr Tyr Asp Tyr Ser Gly |
| | Ala Phe Arg Cys Leu Ala Glu Gly Ala Gly Asp Val Ala Phe Val Lys |
| | His Ser Thr Val Leu Glu Asn Thr Asp Gly Lys Thr Leu Pro Ser Trp |
| | Gly Gln Ala Leu Leu Ser Gln Asp Phe Glu Leu Leu Cys Arg Asp Gly |
| | Ser Arg Ala Asp Val Thr Glu Trp Arg Gln Cys His Leu Ala Arg Val |
| | Pro Ala His Ala Val Val Val Arg Ala Asp Thr Asp Gly Gly Leu Ile Phe |
| | Arg Leu Leu Asn Glu Gly Gln Arg Leu Phe Ser His Glu Gly Ser Ser |
| | Phe Gln Met Phe Ser Ser Glu Ala Tyr Gly Gln Lys Asp Leu Leu Phe |
| | Lys Asp Ser Thr Ser Glu Leu Val Pro Ile Ala Thr Gln Thr Tyr Glu |
| | Ala Trp Leu Gly His Glu Tyr Leu His Ala Met Lys Gly Leu Leu Cys |
| | Asp Pro Asn Arg Leu Pro Pro Tyr Leu Arg Trp Cys Val Leu Ser Thr |
| | Pro Glu Ile Gln Lys Cys Gly Asp Met Ala Val Ala Phe Arg Arg Gln |
| | Arg Leu Lys Pro Glu Ile Gln Cys Val Ser Ala Lys Ser Pro Gln His |
| | Cys Met Glu Arg Ile Gln Ala Glu Gln Val Asp Ala Val Thr Leu Ser |
| | Gly Glu Asp Ile Tyr Thr Ala Gly Lys Thr Tyr Gly Leu Val Pro Ala Ala |
| | Gly Glu His Tyr Ala Pro Glu Asp Ser Ser Asn Ser Tyr Tyr Val Val |
| | Ala Val Val Arg Arg Asp Ser Ser His Ala Phe Thr Leu Asp Glu Leu |
| | Arg Gly Lys Arg Ser Cys His Ala Gly Phe Gly Ser Pro Ala Gly Trp |
| | Asp Val Pro Val Gly Ala Leu Ile Gln Arg Gly Phe Ile Arg Pro Lys Asp |
| | Cys Asp Val Leu Thr Ala Val Ser Glu Phe Phe Asn Ala Ser Cys Val |
| | Pro Val Asn Asn Pro Lys Asn Tyr Pro Ser Ser Leu Cys Ala Leu Cys |
| | Val Gly Asp Glu Gln Gly Arg Asn Lys Cys Val Gly Asn Ser Gln Glu |
| | Arg Tyr Tyr Gly Tyr Arg Gly Ala Phe Arg Cys Leu Val Glu Asn Ala |
| | Gly Asp Val Ala Phe Val Arg His Thr Thr Val Phe Asp Asn Thr Asn |
| | Gly His Asn Ser Glu Pro Trp Ala Ala Glu Leu Arg Ser Glu Asp Tyr |
| | Glu Leu Leu Cys Pro Asn Gly Ala Arg Ala Glu Val Ser Gln Phe Ala |
| | Ala Cys Asn Leu Ala Gln Ile Pro Pro His Ala Val Met Val Arg Pro |
| | Asp Thr Asn Ile Phe Thr Val Tyr Gly Leu Leu Asp Lys Ala Gln Asp |
| | Leu Phe Gly Asp Asp His Asn Lys Asn Gly Phe Lys Met Phe Asp |
| | Ser Ser Asn Tyr His Gly Gln Asp Leu Leu Phe Lys Asp Ala Thr Val |
| | Arg Ala Val Pro Val Gly Glu Lys Thr Thr Tyr Arg Gly Trp Leu Gly |
| | Leu Asp Tyr Val Ala Ala Leu Glu Gly Met Ser Ser Gln Gln Cys Ser |
| | Gly Ala Ala Ala Pro Ala Pro Gly Ala Pro Leu Leu Pro Leu Leu Leu |
| | Pro Ala Leu Ala Ala Arg Leu Leu Pro Pro Ala Leu |
| SEQ ID NO: 2 | Trp Cys Ala Thr Ser Asp Pro Glu Gln His Lys |
| Homo Sapiens | |
| SEQ ID NO: 3 | Arg Ser Ser His Val Thr Ile Asp Thr Leu Lys |
| Homo Sapiens | |
| SEQ ID NO: 4 | Ser Ser His Val Thr Ile Asp Thr Leu Lys Gly Val Lys |
| Homo Sapiens | |
| SEQ ID NO: 5 | Leu Cys Arg Gly Asp Ser Ser Gly Glu Gly Val Cys Asp Lys |
| Homo Sapiens | |
| SEQ ID NO: 6 | Gly Asp Ser Ser Gly Glu Gly Val Cys Asp Lys Ser Pro Leu Glu Arg |
| Homo Sapiens | |
| SEQ ID NO: 7 | Tyr Tyr Asp Tyr Ser Gly Ala Phe Arg |
| Homo Sapiens | |
| SEQ ID NO: 8 | Ala Asp Val Thr Glu Trp Arg |
| Homo Sapiens | |
| SEQ ID NO: 9 | Val Pro Ala His Ala Val Val Val Arg |
| Homo Sapiens | |
| SEQ ID NO: 10 | Ala Asp Thr Asp Gly Gly Leu Ile Phe Arg |
| Homo Sapiens | |
| SEQ ID NO: 11 | Cys Gly Asp Met Ala Val Ala Phe Arg |
| Homo Sapiens | |
| SEQ ID NO: 12 | Leu Lys Pro Glu Ile Gln Cys Val Ser Ala Lys |
| Homo Sapiens | |
| SEQ ID NO: 13 | Asp Ser Ser His Ala Phe Thr Leu Asp Glu Leu Arg |
| Homo Sapiens | |
| SEQ ID NO: 14 | Ser Glu Asp Tyr Glu Leu Leu Cys Pro Asn Gly Ala Arg |
| Homo Sapiens | |
| SEQ ID NO: 15 | Ala Gln Asp Leu Phe Gly Asp Asp His Asn Lys Asn Gly Phe Lys |
| Homo Sapiens | |
| SEQ ID NO: 16 | Phe Ser Ser Glu Ala Tyr Gly Gln Lys Asp Leu Leu Phe Lys Asp Ser |
| Homo Sapiens | Thr Ser Glu Leu Val Pro Ile Ala Thr Gln Thr Tyr Glu Ala Trp Leu Gly |
| | His Glu Tyr Leu His Ala Met |
| SEQ ID NO: 17 | Glu Arg Ile Gln Ala Glu Gln Val Asp Ala Val Thr Leu Ser Gly Glu |
| Homo Sapiens | Asp Ile Tyr Thr Ala Gly Lys Thr Tyr Gly Leu Val Pro Ala Ala Gly Glu |
| | His Tyr Ala Pro Glu Asp Ser Ser Asn Ser Tyr Tyr Val Val Ala Val |
| | Val Arg Arg Asp Ser Ser His Ala Phe Thr Leu Asp Glu Leu Arg Gly |
| | Lys Arg Ser Cys His Ala Gly Phe Gly Ser Pro Ala Gly Trp Asp Val |
| | Pro Val Gly Ala Leu Ile Gln Arg Gly Phe Ile Arg Pro Lys Asp Cys |
| | Asp Val Leu Thr Ala Val Ser Glu Phe Phe Asn Ala Ser Cys Val Pro |
| | Val Asn Asn Pro Lys Asn Tyr Pro Ser Ser Leu Cys Ala Leu Cys Val |
| | Gly Asp Glu Gln Gly Arg Asn Lys Cys Val Gly Asn Ser Gln Glu Arg |
| | Tyr Tyr Gly Tyr Arg Gly Ala Phe Arg Cys Leu Val Glu Asn Ala Gly |
| | Asp Val Ala Phe Val Arg His Thr Thr Val Phe Asp Asn Thr Asn Gly |
| | His Asn Ser Glu Pro Trp Ala Ala Glu Leu Arg Ser Glu Asp Tyr Glu |
| | Leu Leu Cys Pro Asn Gly Ala Arg Ala Glu Val Ser Gln Phe Ala Ala |
| | Cys Asn Leu Ala Gln Ile Pro Pro His Ala Val Met |
| SEQ ID NO: 18 | Val Arg Pro Asp Thr Asn Ile Phe Thr Val Tyr Gly Leu Leu Asp Lys |
| Homo Sapiens | Ala Gln Asp Leu Phe Gly Asp Asp His Asn Lys Asn Gly Phe Lys |
| | Met |
| SEQ ID NO: 19 | Asp Ser Ser Tyr Ser Phe Thr Leu Asp Glu Leu Arg |
| Rattus | |
| norvegicus | |
| domestica and/or | |
| Mus musculus | |

p97 Couplings. As noted above, certain embodiments comprise a p97 polypeptide that is coupled to an agent of interest, for instance, a small molecule, a polypeptide (e.g., peptide, antibody), a peptide mimetic, a peptoid, an aptamer, a detectable entity, or any combination thereof by fusion or conjugation. Also included are conjugates that comprise more than one agent of interest, for instance, a p97 fragment conjugated to an antibody and a small molecule.

Covalent linkages are preferred, however, non-covalent linkages can also be employed, including those that utilize relatively strong non-covalent protein-ligand interactions, such as the interaction between biotin and avidin. Fusion of the p97 fragment with the agent is especially preferred. Operative linkages are also included, which do not necessarily require a directly covalent or non-covalent interaction between the p97 fragment and the agent of interest; examples of such linkages include liposome mixtures that comprise a p97 polypeptide and an agent of interest. Exemplary methods of generating protein conjugates are described herein, and other methods are well-known in the art.

Small Molecules. In particular embodiments, the p97 fragment is conjugated to a small molecule. A “small molecule” refers to an organic compound that is of synthetic or biological origin (biomolecule), but is typically not a polymer. Organic compounds refer to a large class of chemical compounds whose molecules contain carbon, typically excluding those that contain only carbonates, simple oxides of carbon, or cyanides. A “biomolecule” refers generally to an organic molecule that is produced by a living organism, including large polymeric molecules (biopolymers) such as peptides, polysaccharides, and nucleic acids as well, and small molecules such as primary secondary metabolites, lipids, phospholipids, glycolipids, sterols, glycerolipids, vitamins, and hormones. A “polymer” refers generally to a large molecule or macromolecule composed of repeating structural units, which are typically connected by covalent chemical bond.

In certain embodiments, a small molecule has a molecular weight of less than about 1000-2000 Daltons, typically between about 300 and 700 Daltons, and including about 50, 100, 150, 200, 250, 300, 350, 400, 450, 500, 550, 500, 650, 600, 750, 700, 850, 800, 950, 1000 or 2000 Daltons.

Certain small molecules can have the “specific binding” characteristics described for antibodies (infra). For instance, a small molecule can specifically bind to a target described herein with a binding affinity (Kd) of at least about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM. In certain embodiments a small specifically binds to a cell surface receptor or other cell surface protein.

Polypeptide Agents. In particular embodiments, the agent of interest is a peptide or polypeptide, or fragment thereof. The terms “peptide” and “polypeptide” are used interchangeably herein, however, in certain instances, the term “peptide” can refer to shorter polypeptides, for example, polypeptides that consist of about 2, 3, 4, 5, 6, 7, 8, 9, 10, 11, 12, 13, 14, 15, 16, 17, 18, 19, 20, 25, 30, 35, 40, 45, or 50 amino acids, including all integers and ranges (e.g., 5-10, 8-12, 10-15) in between. Polypeptides and peptides can be composed of naturally-occurring amino acids and/or non-naturally occurring amino acids, as described herein. Antibodies are also included as polypeptides. Fragments or portion of these peptides, polypeptides, or antigens are contemplated as within the scope of the present invention.

Exemplary polypeptide agents include polypeptides associated with lysosomal storage disorders. Examples of such polypeptides include aspartylglucosaminidase, acid lipase, cysteine transporter, Lamp-2, α-galactosidase A, acid ceramidase, α-L-fucosidase, β-hexosaminidase A, GM2-ganglioside activator (GM2A), α-D-mannosidase, β-D-mannosidase, arylsulfatase A, saposin B, neuraminidase, α-N-acetylglucosaminidase phosphotransferase, phosphotransferase γ-subunit, L-iduronidase, iduronate-2-sulfatase, heparan-N-sulfatase, α-N-acetylglucosaminidase, acetylCoA:N-acetyltransferase, N-acetylglucosamine 6-sulfatase, galactose 6-sulfatase, β-galactosidase, N-acetylgalactosamine 4-sulfatase, hyaluronoglucosaminidase, sulfatases, palmitoyl protein thioesterase, tripeptidyl peptidase I, acid sphingomyelinase, cathepsin A, cathepsin K, α-galactosidase B, NPC1, NPC2, sialin, and sialic acid transporter, including fragments, variants, and derivatives thereof. Certain embodiments include polypeptides such as imiglucerase, β-glucocerebrosidase, velaglucerase alfa, taliglucerase alfa, eliglustat, miglustat, which are often used for the treatment of Gaucher disease due to GBA1 mutations.

Certain embodiments include polypeptides such as interferon-β polypeptides, such as interferon-β1a (e.g., AVONEX, REBIF) and interferon-β1b (e.g., Betaseron), which are often used for the treatment of multiple sclerosis (MS).

In some embodiments, as noted above, the polypeptide agent is an antibody or an antigen-binding fragment thereof. The antibody or antigen-binding fragment used in the conjugates or compositions of the present invention can be of essentially any type. Particular examples include therapeutic and diagnostic antibodies. As is well known in the art, an antibody is an immunoglobulin molecule capable of specific binding to a target, such as a carbohydrate, polynucleotide, lipid, polypeptide, etc., through at least one epitope recognition site, located in the variable region of the immunoglobulin molecule.

As used herein, the term “antibody” encompasses not only intact polyclonal or monoclonal antibodies, but also fragments thereof (such as dAb, Fab, Fab′, F(ab′h, Fv), single chain (ScFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity.

In some embodiments, the antibody or antigen-binding fragment or other polypeptide specifically binds to a cell surface receptor or other cell surface protein. In some embodiments, the antibody or antigen-binding fragment or other polypeptide specifically binds to a ligand of a cell surface receptor or other cell surface protein. In some embodiments, the antibody or antigen-binding fragment or other polypeptide specifically binds to an intracellular protein.

Antibodies or polypeptides may be prepared by any of a variety of techniques known to those of ordinary skill in the art. See, e.g., Harlow and Lane, Antibodies: A Laboratory Manual, Cold Spring Harbor Laboratory, 1988. Monoclonal antibodies specific for a polypeptide of interest may be prepared, for example, using the technique of Kohler and Milstein, Eur. J. Immunol. 6:511-519, 1976, and improvements thereto. Also included are methods that utilize transgenic animals such as mice to express human antibodies. See, e.g., Neuberger et al., Nature Biotechnology 14:826, 1996; Lonberg et al., Handbook of Experimental Pharmacology 113:49-101, 1994; and Lonberg et al., Internal Review of Immunology 13:65-93, 1995.

Antibodies or polypeptides can also be generated or identified by the use of phage display or yeast display libraries (see, e.g., U.S. Pat. No. 7,244,592; Chao et al., Nature Protocols. 1:755-768, 2006). Non-limiting examples of available libraries include cloned or synthetic libraries, such as the Human Combinatorial Antibody Library (HuCAL), in which the structural diversity of the human antibody repertoire is represented by seven heavy chain and seven light chain variable region genes. The combination of these genes gives rise to 49 frameworks in the master library. By superimposing highly variable genetic cassettes (CDRs=complementarity determining regions) on these frameworks, the vast human antibody repertoire can be reproduced. Also included are human libraries designed with human-donor-sourced fragments encoding a light-chain variable region, a heavy-chain CDR-3, synthetic DNA encoding diversity in heavy-chain CDR-1, and synthetic DNA encoding diversity in heavy-chain CDR-2.

Other libraries suitable for use will be apparent to persons skilled in the art. The p97 polypeptides described herein and known in the art may be used in the purification process in, for example, an affinity chromatography step.

In certain embodiments, the antibodies or polypeptides provided herein may take the form of a nanobody. Minibodies are encoded by single genes and are efficiently produced in almost all prokaryotic and eukaryotic hosts, for example, E. coli (see U.S. Pat. No. 6,765,087), moulds (for example Aspergillus or Trichoderma) and yeast (for example Saccharomyces, Kluyvermyces, Hansenula or Pichia (see U.S. Pat. No. 6,838,254). The production process is scalable and multi-kilogram quantities of nanobodies have been produced. Nanobodies may be formulated as a ready-to-use solution having a long shelf life. The Nanoclone method (see WO 06/079372) is a proprietary method for generating Nanobodies against a desired target, based on automated high-throughput selection of B-cells.

In certain embodiments, the antibodies or antigen-binding fragments thereof or polypeptides are humanized. These embodiments refer to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin.

In certain embodiments, the antibodies of the present invention may be chimeric antibodies. In this regard, a chimeric antibody is comprised of an antigen-binding fragment of an antibody operably linked or otherwise fused to a heterologous Fe portion of a different antibody. In certain embodiments, the heterologous Fe domain is of human origin. In other embodiments, the heterologous Fe domain may be from a different Ig class from the parent antibody, including IgA (including subclasses IgAI and IgA2), IgD, IgE, IgG (including subclasses IgGI, IgG2, IgG3, and IgG4), and IgM. In further embodiments, the heterologous Fe domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

Peptide Mimetics. Certain embodiments employ “peptide mimetics.” Peptide analogs are commonly used in the pharmaceutical industry as non-peptide drugs with properties analogous to those of the template peptide. These types of non-peptide compound are termed “peptide mimetics” or “peptidomimetics” (Luthman et al., A Textbook of Drug Design and Development, 14:386-406, 2nd Ed., Harwood Academic Publishers, 1996; Joachim Grante, Angew. Chem. Int. Ed. Engl., 33:1699-1720, 1994; Fauchere, Adv. Drug Res., 15:29, 1986; Veber and Freidinger TINS, p. 392 (1985); and Evans et al., J. Med. Chem. 30:229, 1987). A peptidomimetic is a molecule that mimics the biological activity of a peptide but is no longer peptidic in chemical nature. Peptidomimetic compounds are known in the art and are described, for example, in U.S. Pat. No. 6,245,886.

A peptide mimetic can have the “specific binding” characteristics described for antibodies (supra). For example, a peptide mimetic can specifically bind to a target described herein with a binding affinity (Kd) of at least about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM. In some embodiments a peptide mimetic specifically binds to a cell surface receptor or other cell surface protein. In some embodiments, the peptide mimetic specifically binds to at least one cancer-associated antigen described herein. In particular embodiments, the peptide mimetic specifically binds to at least one nervous system-associated, pain-associated, and/or autoimmune-associated antigen described herein.

Peptoids. The conjugates of the present invention also includes “peptoids.” Peptoid derivatives of peptides represent another form of modified peptides that retain the important structural determinants for biological activity, yet eliminate the peptide bonds, thereby conferring resistance to proteolysis (Simon, et al., PNAS USA. 89:9367-9371, 1992). Peptoids are oligomers of N-substituted glycines. A number of N-alkyl groups have been described, each corresponding to the side chain of a natural amino acid. The peptidomimetics of the present invention include compounds in which at least one amino acid, a few amino acids or all amino acid residues are replaced by the corresponding N-substituted glycines. Peptoid libraries are described, for example, in U.S. Pat. No. 5,811,387.

A peptoid can have the “specific binding” characteristics described for antibodies (supra). For instance, a peptoid can specifically bind to a target described herein with a binding affinity (Kd) of at least about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM. In certain embodiments a peptoid specifically binds to a cell surface receptor or other cell surface protein. In some embodiments, the peptoid specifically binds to at least one cancer-associated antigen described herein. In particular embodiments, the peptoid specifically binds to at least one nervous system-associated, pain-associated, and/or autoimmune-associated antigen described herein.

Aptamers. The p97 conjugates of the present invention also include aptamers (see, e.g., Ellington et al., Nature. 346, 818-22, 1990; and Tuerk et al., Science. 249, 505-10, 1990). Examples of aptamers include nucleic acid aptamers (e.g., DNA aptamers, RNA aptamers) and peptide aptamers. Nucleic acid aptamers refer generally to nucleic acid species that have been engineered through repeated rounds of in vitro selection or equivalent method, such as SELEX (systematic evolution of ligands by exponential enrichment), to bind to various molecular targets such as small molecules, proteins, nucleic acids, and even cells, tissues and organisms. See, e.g., U.S. Pat. Nos. 6,376,190; and 6,387,620.

Peptide aptamers typically include a variable peptide loop attached at both ends to a protein scaffold, a double structural constraint that typically increases the binding affinity of the peptide aptamer to levels comparable to that of an antibody's (e.g., in the nanomolar range). In certain embodiments, the variable loop length may be composed of about 10-20 amino acids (including all integers in between), and the scaffold may include any protein that has good solubility and compacity properties. Certain exemplary embodiments may utilize the bacterial protein Thioredoxin-A as a scaffold protein, the variable loop being inserted within the reducing active site (-Cys-Gly-Pro-Cys-loop in the wild protein), with the two cysteines lateral chains being able to form a disulfide bridge. Methods for identifying peptide aptamers are described, for example, in U.S. Application No. 2003/0108532.

An aptamer can have the “specific binding” characteristics described for antibodies (supra). For instance, an aptamer can specifically bind to a target described herein with a binding affinity (Kd) of at least about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM. In particular embodiments, an aptamer specifically binds to a cell surface receptor or other cell surface protein. In some embodiments, the aptamer specifically binds to at least one cancer-associated antigen described herein. In particular embodiments, the aptamer specifically binds to at least one nervous system-associated, pain-associated, and/or autoimmune-associated antigen described herein.

The particular active agent that is suitable for treating Lewy body dementias can be any agent, including those small molecules, polypeptide agents, peptide mimetics, peptoids, aptamers, as well as enzymes such as currently being used to treat Gaucher disease caused by GBA1 mutations. Some examples of active agents currently available to treat Gaucher disease are set forth below, although other active agents not specifically identified herein are intended to be included within the scope of the invention.

Glucocerebrosidase

The abnormal build-up of alpha-synuclein that is characteristic of Lewy bodies is also correlated with a decrease in the enzyme glucocerebrosidase and abnormal brain levels of the sphingolipid glucocerebroside. Glucocerebrosidase (which is also known as beta-glucosylcerebrosidase, β-glucosylcerebrosidase, or by the abbreviation GCase), is a lysosomal enzyme involved in the breakdown of glucocerebroside (also known as glucosylceramide). Glucocerebroside is needed for normal neuron function.

The present invention provides methods and compositions for delivering the enzyme glucocerebrosidase.

Cerezyme (imiglucerase) is an analogue of the human enzyme ß-glucocerebrosidase, produced by recombinant DNA technology. ß-Glucocerebrosidase (ß-D-glucosyl-N-acylsphingosine glucohydrolase, E.C. 3.2.1.45) is a lysosomal glycoprotein enzyme which catalyzes the h to glucose and ceramide. Cerezyme is produced by recombinant DNA technology using mammalian cell culture (Chinese hamster ovary). Purified imiglucerase is a monomeric glycoprotein of 497 amino acids, containing 4 N-linked glycosylation sites (Mr=60,430). Imiglucerase differs from placental glucocerebrosidase by one amino acid at position 495, where histidine is substituted for arginine. The oligosaccharide chains at the glycosylation sites have been modified to terminate in mannose sugars. The modified carbohydrate structures on imiglucerase are somewhat different from those on placental glucocerebrosidase. These mannose-terminated oligosaccharide chains of imiglucerase are specifically recognized by endocytic carbohydrate receptors on macrophages, the cells that accumulate lipid in Gaucher disease.

Further information on Cerezyme (imiglucerase) includes the following: chemical Name: 495-L-Histidineglucosylceramidase (human placenta isoenzyme protein moiety). Chemical formula: C₂₅₃₂H₃₈₄₃N₆₇₁O₇₁₁S₁₆. CAS Number: 154248-97-2.

VPRIV (velaglucerase alfa), VPRIV is indicated for long-term enzyme replacement therapy (ERT) for patients with type 1 Gaucher disease. The active ingredient of VPRIV is velaglucerase alfa, which is produced by gene activation technology in a human fibroblast cell line. Velaglucerase alfa is a glycoprotein of 497 amino acids; with a molecular weight of approximately 63 kDa. Velaglucerase alfa has the same amino acid sequence as the naturally occurring human enzyme, glucocerebrosidase. Velaglucerase alfa contains 5 potential N-linked glycosylation sites; four of these sites are occupied by glycan chains. Velaglucerase alfa contains predominantly high mannose-type N-linked glycan chains. The high mannose-type N-linked glycan chains are specifically recognized and internalized via the mannose receptor present on the surface on macrophages, the cells that accumulate glucocerebroside in Gaucher disease. Velaglucerase alfa catalyzes the hydrolysis of the glycolipid glucocerebroside to glucose and ceramide in the lysosome.

Further information on VPRIV (velaglucerase alfa) includes the following: Chemical formula C₂₅₃₂H₃₈₅₀N₆₇₂O₇₁₁S₁₆. CAS Number 884604-91-5.

ELELYSO (taliglucerase alfa) is a hydrolytic lysosomal glucocerebroside-specific enzyme indicated for the treatment of patients with a confirmed diagnosis of Type 1 Gaucher disease. Taliglucerase alfa, a hydrolytic lysosomal glucocerebroside-specific enzyme for intravenous infusion, is a recombinant active form of the lysosomal enzyme, β-glucocerebrosidase, which is expressed in genetically modified carrot plant root cells cultured in a disposable bioreactor system (ProCellEx®). β-Glucocerebrosidase (β-D-glucosyl-N-acylsphingosine glucohydrolase, E.C. 3.2.1.45) is a lysosomal glycoprotein enzyme that catalyzes the hydrolysis of the glycolipid glucocerebroside to glucose and ceramide. ELELYSO is produced by recombinant DNA technology using plant cell culture (carrot). Purified taliglucerase alfa is a monomeric glycoprotein containing 4 N-linked glycosylation sites (Mr=60,800). Taliglucerase alfa differs from native human glucocerebrosidase by two amino acids at the N terminal and up to 7 amino acids at the C terminal. Taliglucerase alfa is a glycosylated protein with oligosaccharide chains at the glycosylation sites having terminal mannose sugars. These mannose-terminated oligosaccharide chains of taliglucerase alfa are specifically recognized by endocytic carbohydrate receptors on macrophages, the cells that accumulate lipid in Gaucher disease.

Further information on Elelyso (taliglucerase alfa) includes the following: Chemical formula C₂₅₈₀H₃₉₁₈N₆₈₀O₇₂₇S₁₇. CAS Number 37228-64-1.

The previously marketed drug CEREDASE (Alglucerase) is also useful herein. CAS ID: 37228-64-1. Ceredase is the trade name of a citrate buffered solution of alglucerase that was manufactured by Genzyme Corporation from human placental tissue. However, the other forms of Beta-glucocerebrosidase have generally replaced the use of alglucerase.

Pharmakokinetic and pharmacodynamic considerations: When administered directly by intravenous means, the target Beta-glucocerebrosidase would generally have an AUC (area under the curve) (from zero to infinity, AUC(0 to ∞) from about 500 to about 25,000 ng*h/ml, or preferably from about 1000 to about 10,000 ng*h/ml, or about 1500 to about 7500 ng*h/ml. However, with the methods and compositions of the present invention the Beta-glucocerebrosidase is administered for more efficient and selective transport across the blood-brain barrier, thus providing the opportunity for having a lower systemic exposure with less potential for adverse reactions and other desired side effects or toxicity.

Glucosylcermide Synthase Inhibitors

CERDELGA™ (eliglustat) is a glucosylceramide synthase inhibitor indicated for the long-term treatment of adult patients with Gaucher disease type 1 who are CYP2D6 intermediate metabolizers (IMs), or poor metabolizers (PMs) as detected by an FDA-cleared test. CERDELGA (eliglustat) capsules contain eliglustat tartrate, which is a small molecule inhibitor of glucosylceramide synthase that resembles the ceramide substrate for the enzyme, with the chemical name N-((1R,2R)-1-(2,3-dihydrobenzo[b][1,4]dioxin-6-yl)-1-hydroxy-3-(pyrrolidin-1-yl)propan-2-yl)octanamide (2R,3R)-2,3-dihydroxysuccinate.

ZAVESCA (miglustat) is a glucosylceramide synthase inhibitor indicated as monotherapy for the treatment of mild/moderate type 1 Gaucher disease for whom enzyme replacement therapy is not a therapeutic option. Miglustat is an inhibitor of the enzyme glucosylceramide synthase, which is a glucosyl transferase enzyme responsible for the first step in the synthesis of most glycosphingolipids. Zavesca is an N-alkylated imino sugar, a synthetic analog of D-glucose. The chemical name for miglustat is 1,5-(butylimino)-1,5-dideoxy-D-glucitol.

Detectable Entities. In some embodiments, the p97 fragment is conjugated to a “detectable entity.” Exemplary detectable entities include, without limitation, iodine-based labels, radioisotopes, fluorophores/fluorescent dyes, and nanoparticles. The detectable entity may be present on the active agent.

Exemplary iodine-based labels include diatrizoic acid (Hypaque®, GE Healthcare) and its anionic form, diatrizoate. Diatrizoic acid is a radio-contrast agent used in advanced X-ray techniques such as CT scanning. Also included are iodine radioisotopes, described below.

Exemplary radioisotopes that can be used as detectable entities include ³²P, ³³P, ³⁵S, ³H, ¹⁸F, ¹¹C, ¹³N, ¹⁵O, ¹¹¹N, ¹⁶⁹Yb, ^{99m}TC, ⁵⁵Fe and isotopes of iodine such as ¹²³I, ¹²⁴I, ¹²⁵I, and ¹³¹I. These radioisotopes have different half-lives, types of decay, and levels of energy which can be tailored to match the needs of a particular protocol. Certain of these radioisotopes can be selectively targeted or better targeted to CNS tissues by conjugation to p97 polypeptides, for instance, to improve the medical imaging of such tissues. Examples of fluorophores or fluorochromes that can be used as directly detectable entities include fluorescein, tetramethylrhodamine, Texas Red, Oregon Green®, and a number of others (e.g., Haugland, Handbook of Fluorescent Probes—9th Ed., 2002, Malec. Probes, Inc., Eugene Oreg.; Haugland, The Handbook: A Guide to Fluorescent Probes and Labeling Technologies—10th Ed., 2005, Invitrogen, Carlsbad, Calif.). Also included are light-emitting or otherwise detectable dyes. The light emitted by the dyes can be visible light or invisible light, such as ultraviolet or infrared light. In exemplary embodiments, the dye may be a fluorescence resonance energy transfer (FRET) dye; a xanthene dye, such as fluorescein and rhodamine; a dye that has an amino group in the alpha or beta position (such as a naphthylamine dye, 1-dimethylaminonaphthyl-5-sulfonate, 1-anilino-8-naphthalende sulfonate and 2-p-touidinyl-6-naphthalene sulfonate); a dye that has 3-phenyl-7-isocyanatocoumarin; an acridine, such as 9-isothiocyanatoacridine and acridine orange; a pyrene, a bensoxadiazole and a stilbene; a dye that has 3-(s-carboxypentyl)-3′-ethyl-5,5′-dimethyloxacarbocyanine (CYA); 6-carboxy fluorescein (FAM); 5&6-carboxyrhodamine-110 (R110); 6-carboxyrhodamine-6G (R6G); N,N,N′,N′-tetramethyl-6-carboxyrhodamine (TAM RA); 6-carboxy-X-rhodamine (ROX); 6-carboxy-4′,5′-dichloro-2′,7′-dimethoxyfluorescein (JOE); ALEXA FLUOR™; Cy2; Texas Red and Rhodamine Red; 6-carboxy-2′,4,7,7′-tetrachlorofluorescein (TET); 6-carboxy-2′,4,4′,5′,7,7′-hexachlorofluorescein (HEX); 5-carboxy-2′,4′,5′,7′-tetrachlorofluorescein (ZOE); NAN; NED; Cy3; Cy3.5; Cy5; Cy5.5; Cy7; and Cy7.5; IR800CW, ICG, Alexa Fluor 350; Alexa Fluor 488; Alexa Fluor 532; Alexa Fluor 546; Alexa Fluor 568; Alexa Fluor 594; Alexa Fluor 647; Alexa Fluor 680, or Alexa Fluor 750. Certain embodiments include conjugation to chemotherapeutic agents (e.g., paclitaxel, adriamycin) that are labeled with a detectable entity, such as a fluorophore (e.g., Oregon Green®, Alexa Fluor 488).

Nanoparticles usually range from about 1-1000 nm in size and include diverse chemical structures such as gold and silver particles and quantum dots. When irradiated with angled incident white light, silver or gold nanoparticles ranging from about 40-120 nm will scatter monochromatic light with high intensity. The wavelength of the scattered light is dependent on the size of the particle. Four to five different particles in close proximity will each scatter monochromatic light, which when superimposed will give a specific, unique color. Derivatized nanoparticles such as silver or gold particles can be attached to a broad array of molecules including, proteins, antibodies, small molecules, receptor ligands, and nucleic acids. Specific examples of nanoparticles include metallic nanoparticles and metallic nanoshells such as gold particles, silver particles, copper particles, platinum particles, cadmium particles, composite particles, gold hollow spheres, gold-coated silica nanoshells, and silica-coated gold shells. Also included are silica, latex, polystyrene, polycarbonate, polyacrylate, PVDF nanoparticles, and colored particles of any of these materials.

Quantum dots are fluorescing crystals about 1-5 nm in diameter that are excitable by light over a large range of wavelengths. Upon excitation by light having an appropriate wavelength, these crystals emit light, such as monochromatic light, with a wavelength dependent on their chemical composition and size. Quantum dots such as CdSe, ZnSe, InP, or InAs possess unique optical properties; these and similar quantum dots are available from a number of commercial sources (e.g., NN-Labs, Fayetteville, Ark.; Ocean Nanotech, Fayetteville, Ark.; Nanoco Technologies, Manchester, UK; Sigma-Aldrich, St. Louis, Mo.).

Polypeptide Variants and Fragments. Certain embodiments include variants and/or fragments of the reference polypeptides described herein, whether described by name or by reference to a sequence identifier, including p97 polypeptides and polypeptide-based agents such as antibodies. The wild-type or most prevalent sequences of these polypeptides are known in the art, and can be used as a comparison for the variants and fragments described herein.

A polypeptide “variant,” as the term is used herein, is a polypeptide that typically differs from a polypeptide specifically disclosed herein by one or more substitutions, deletions, additions and/or insertions. Variant polypeptides are biologically active, that is, they continue to possess the enzymatic or binding activity of a reference polypeptide. Such variants may result from, for example, genetic polymorphism and/or from human manipulation.

In many instances, a biologically active variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics. When it is desired to alter the amino acid sequence of a polypeptide to create an equivalent, or even an improved, variant or portion of a polypeptide of the invention, one skilled in the art will typically change one or more of the codons of the encoding DNA sequence according to Table A below.

Certain embodiments include variants and/or fragments of the reference polypeptides described herein, whether described by name or by reference to a sequence identifier, including p97 polypeptides and GBA1-related proteins. The wild-type or most prevalent sequences of these polypeptides are known in the art, and can be used as a comparison for the variants and fragments described herein.

A polypeptide “variant,” as the term is used herein, is a polypeptide that typically differs from a polypeptide specifically disclosed herein by one or more substitutions, deletions, additions and/or insertions. Variant polypeptides are biologically active, that is, they continue to possess the enzymatic or binding activity of a reference polypeptide. Such variants may result from, for example, genetic polymorphism and/or from human manipulation.

In many instances, a biologically active variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics. When it is desired to alter the amino acid sequence of a polypeptide to create an equivalent, or even an improved, variant or portion of a polypeptide of the invention, one skilled in the art will typically change one or more of the codons of the encoding DNA sequence according to Table A below.

TABLE A

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their utility.

In making such changes, the hydropathic index of amino acids may be considered. The importance of the hydropathic amino acid index in conferring interactive biologic function on a protein is generally understood in the art (Kyte & Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte & Doolittle, 1982). These values are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); praline (−1.6); histidine (−3.2); glutamate (−3.5); glutamine (−3.5); aspartate (−3.5); asparagine (−3.5); lysine (−3.9); and arginine (−4.5). It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred.

It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); praline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his.

A variant may also, or alternatively, contain non-conservative changes. In a preferred embodiment, variant polypeptides differ from a native sequence by substitution, deletion or addition of fewer than about 10, 9, 8, 7, 6, 5, 4, 3, 2 amino acids, or even 1 amino acid. Variants may also (or alternatively) be modified by, for example, the deletion or addition of amino acids that have minimal influence on the immunogenicity, secondary structure, enzymatic activity, and/or hydropathic nature of the polypeptide.

In certain embodiments, variants of the DSSHAFTLDELR (SEQ ID NO: 2) can be based on the sequence of p97 sequences from other organisms, as shown in Table B of U.S. Pat. No. 9,364,567, issued Jun. 14, 2016, the entire contents of such patent is hereby incorporated by reference as if set out in full.

In general, variants will display at least about 30%, 40%, 50%, 55%, 60%, 65%, 70%, 75%, 80%, 85%, 90%, 91%, 92%, 93%, 94%, 95%, 96%, 97%, 98%, 99% similarity or sequence identity or sequence homology to a reference polypeptide sequence. Moreover, sequences differing from the native or parent sequences by the addition (e.g., (-terminal addition, N-terminal addition, both), deletion, truncation, insertion, or substitution of about 1, 2, 3, 4, 5, 6, 7, 8, 9, 10, 11, 12, 13, 14, 15, 16, 17, 18, 19, 20, 30, 40, 50, 60, 70, 80, 90, 100 or more amino acids but which retain the properties or activities of a parent or reference polypeptide sequence are contemplated.

In some embodiments, variant polypeptides differ from reference sequence by at least one but by less than 50, 40, 30, 20, 15, 10, 8, 6, 5, 4, 3 or 2 amino acid residue(s). In other embodiments, variant polypeptides differ from a reference sequence by at least 1% but less than 20%, 15%, 10% or 5% of the residues. (If this comparison requires alignment, the sequences should be aligned for maximum similarity. “Looped” out sequences from deletions or insertions, or mismatches, are considered differences.)

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch, (J. Mol. Biol. 48: 444-453, 1970) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna. CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (Cabios. 4:11-17, 1989) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (Nucleic Acids Res. 25: 3389-3402, 1997). When utilizing BLAST and Gapped BLAST programs, the default parameters of the respective programs (e.g., XBLAST and NBLAST) can be used.

In one embodiment, as noted above, polynucleotides and/or polypeptides can be evaluated using a BLAST alignment tool. A local alignment consists simply of a pair of sequence segments, one from each of the sequences being compared. A modification of Smith-Waterman or Sellers algorithms will find all segment pairs whose scores cannot be improved by extension or trimming, called high-scoring segment pairs (HSPs). The results of the BLAST alignments include statistical measures to indicate the likelihood that the BLAST score can be expected from chance alone.

The raw score, S, is calculated from the number of gaps and substitutions associated with each aligned sequence wherein higher similarity scores indicate a more significant alignment. Substitution scores are given by a look-up table (see PAM, BLOSUM).

Gap scores are typically calculated as the sum of G, the gap opening penalty and L, the gap extension penalty. For a gap of length n, the gap cost would be G+Ln. The choice of gap costs, G and Lis empirical, but it is customary to choose a high value for G (10-15), e.g., 11, and a low value for L (1-2) e.g., 1.

The bit score, S′, is derived from the raw alignment score S in which the statistical properties of the scoring system used have been taken into account. Bit scores are normalized with respect to the scoring system, therefore they can be used to compare alignment scores from different searches. The terms “bit score” and “similarity score” are used interchangeably. The bit score gives an indication of how good the alignment is; the higher the score, the better the alignment.

The E-Value, or expected value, describes the likelihood that a sequence with a similar score will occur in the database by chance. It is a prediction of the number of different alignments with scores equivalent to or better than S that are expected to occur in a database search by chance. The smaller the E-Value, the more significant the alignment. For example, an alignment having an E value of e-117 means that a sequence with a similar score is very unlikely to occur simply by chance. Additionally, the expected score for aligning a random pair of amino acids is required to be negative, otherwise long alignments would tend to have high score independently of whether the segments aligned were related. Additionally, the BLAST algorithm uses an appropriate substitution matrix, nucleotide or amino acid and for gapped alignments uses gap creation and extension penalties. For example, BLAST alignment and comparison of polypeptide sequences are typically done using the BLOSUM62 matrix, a gap existence penalty of 11 and a gap extension penalty of 1.

In one embodiment, sequence similarity scores are reported from BLAST analyses done using the BLOSUM62 matrix, a gap existence penalty of 11 and a gap extension penalty of 1.

In a particular embodiment, sequence identity/similarity scores provided herein refer to the value obtained using GAP Version 10 (GCG, Accelrys, San Diego, Calif.) using the following parameters:% identity and % similarity for a nucleotide sequence using GAP Weight of 50 and Length Weight of 3, and the nwsgapdna.cmp scoring matrix;% identity and % similarity for an amino acid sequence using GAP Weight of 8 and Length Weight of 2, and the BLOSUM62 scoring matrix (Henikoff and Henikoff, PNAS USA. 89:10915-10919, 1992). GAP uses the algorithm of Needleman and Wunsch (J Mol Biol. 48:443-453, 1970) to find the alignment of two complete sequences that maximizes the number of matches and minimizes the number of gaps.

As noted above, a reference polypeptide may be altered in various ways including amino acid substitutions, deletions, truncations, additions, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (PNAS USA. 82: 488-492, 1985); Kunkel et al., (Methods in Enzymol. 154: 367-382, 1987), U.S. Pat. No. 4,873,192, Watson, J. D. et al., (“Molecular Biology of the Gene,” Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

Methods for screening gene products of combinatorial libraries made by such modifications, and for screening cDNA libraries for gene products having a selected property are known in the art. Such methods are adaptable for rapid screening of the gene libraries generated by combinatorial mutagenesis of reference polypeptides. As one example, recursive ensemble mutagenesis (REM), a technique which enhances the frequency of functional mutants in the libraries, can be used in combination with the screening assays to identify polypeptide variants (Arkin and Yourvan, PNAS USA 89: 7811-7815, 1992; Delgrave et al., Protein Engineering. 6: 327-331, 1993).

Exemplary Methods for Conjugation. Conjugation or coupling of a p97 polypeptide sequence to an agent of interest can be carried out using standard chemical, biochemical and/or molecular techniques. Indeed, it will be apparent how to make a p97 conjugate in light of the present disclosure using available art-recognized methodologies. Of course, it will generally be preferred when coupling the primary components of a p97 conjugate of the present invention that the techniques employed and the resulting linking chemistries do not substantially disturb the desired functionality or activity of the individual components of the conjugate.

The particular coupling chemistry employed will depend upon the structure of the biologically active agent (e.g., small molecule, polypeptide), the potential presence of multiple functional groups within the biologically active agent, the need for protection/deprotection steps, chemical stability of the agent, and the like, and will be readily determined by one skilled in the art. Illustrative coupling chemistry useful for preparing the p97 conjugates of the invention can be found, for example, in Wong (1991), “Chemistry of Protein Conjugation and Crosslinking”, CRC Press, Boca Raton, Fla.; and Brinkley “A Brief Survey of Methods for Preparing Protein Conjugates with Dyes, Haptens, and Crosslinking Reagents,” in Bioconjug. Chem., 3:2013, 1992. Preferably, the binding ability and/or activity of the conjugate is not substantially reduced as a result of the conjugation technique employed, for example, relative to the unconjugated agent or the unconjugated p97 polypeptide.

In certain embodiments, a p97 polypeptide sequence may be coupled to an agent of interest either directly or indirectly. A direct reaction between a p97 polypeptide sequence and an agent of interest is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to indirectly couple a p97 polypeptide sequence and an agent of interest via a linker group, including non-peptide linkers and peptide linkers. A linker group can also function as a spacer to distance an agent of interest from the p97 polypeptide sequence in order to avoid interference with binding capabilities, targeting capabilities or other functionalities. A linker group can also serve to increase the chemical reactivity of a substituent on an agent, and thus increase the coupling efficiency. An increase in chemical reactivity may also facilitate the use of agents, or functional groups on agents, which otherwise would not be possible. The selection of releasable or stable linkers can also be employed to alter the pharmacokinetics of a p97 conjugate and attached agent of interest. Illustrative linking groups include, for example, disulfide groups, thioether groups, acid labile groups, photolabile groups, peptidase labile groups and esterase labile groups. In other illustrative embodiments, the conjugates include linking groups such as those disclosed in U.S. Pat. No. 5,208,020 or EP Patent O 425 235 BI, and Chari et al., Cancer Research. 52: 127-131, 1992. Additional exemplary linkers are described below.

In some embodiments, it may be desirable to couple more than one p97 polypeptide sequence to an agent, or vice versa. For example, in certain embodiments, multiple p97 polypeptide sequences are coupled to one agent, or alternatively, one or more p97 polypeptides are conjugated to multiple agents. The p97 polypeptide sequences can be the same or different. Regardless of the particular embodiment, conjugates containing multiple p97 polypeptide sequences may be prepared in a variety of ways. For example, more than one polypeptide may be coupled directly to an agent, or linkers that provide multiple sites for attachment can be used. Any of a variety of known heterobifunctional crosslinking strategies can be employed for making conjugates of the invention. It will be understood that many of these embodiments can be achieved by controlling the stoichiometries of the materials used during the conjugation/crosslinking procedure.

In certain exemplary embodiments, a reaction between an agent comprising a succinimidyl ester functional group and a p97 polypeptide comprising an amino group forms an amide linkage; a reaction between an agent comprising a oxycarbonylimidizaole functional group and a P97 polypeptide comprising an amino group forms an carbamate linkage; a reaction between an agent comprising a p-nitrophenyl carbonate functional group and a P97 polypeptide comprising an amino group forms an carbamate linkage; a reaction between an agent comprising a trichlorophenyl carbonate functional group and a P97 polypeptide comprising an amino group forms an carbamate linkage; a reaction between an agent comprising a thio ester functional group and a P97 polypeptide comprising an n-terminal amino group forms an amide linkage; a reaction between an agent comprising a proprionaldehyde functional group and a P97 polypeptide comprising an amino group forms a secondary amine linkage.

In some exemplary embodiments, a reaction between an agent comprising a butyraldehyde functional group and a P97 polypeptide comprising an amino group forms a secondary amine linkage; a reaction between an agent comprising an acetal functional group and a P97 polypeptide comprising an amino group forms a secondary amine linkage; a reaction between an agent comprising a piperidone functional group and a P97 polypeptide comprising an amino group forms a secondary amine linkage; a reaction between an agent comprising a methylketone functional group and a P97 polypeptide comprising an amino group forms a secondary amine linkage; a reaction between an agent comprising a tresylate functional group and a P97 polypeptide comprising an amino group forms a secondary amine linkage; a reaction between an agent comprising a maleimide functional group and a P97 polypeptide comprising an amino group forms a secondary amine linkage; a reaction between an agent comprising a aldehyde functional group and a P97 polypeptide comprising an amino group forms a secondary amine linkage; and a reaction between an agent comprising a hydrazine functional group and a P97 polypeptide comprising an carboxylic acid group forms a secondary amine linkage.

In particular exemplary embodiments, a reaction between an agent comprising a maleimide functional group and a P97 polypeptide comprising a thiol group forms a thio ether linkage; a reaction between an agent comprising a vinyl sulfone functional group and a P97 polypeptide comprising a thiol group forms a thio ether linkage; a reaction between an agent comprising a thiol functional group and a P97 polypeptide comprising a thiol group forms a di-sulfide linkage; a reaction between an agent comprising a orthopyridyl disulfide functional group and a P97 polypeptide comprising a thiol group forms a di-sulfide linkage; and a reaction between an agent comprising an iodoacetamide functional group and a P97 polypeptide comprising a thiol group forms a thio ether linkage.

In a specific embodiment, an amine-to-sulfhydryl crosslinker is used for preparing a conjugate.

In one preferred embodiment, for example, the crosslinker is succinimidyl-4-(N-maleimidomethyl)cyclohexane-1-carboxylate (SMCC) (Thermo Scientific), which is a sulfhydryl crosslinker containing NHS-ester and maleimide reactive groups at opposite ends of a medium-length cyclohexane-stabilized spacer arm (8.3 angstroms). SMCC is a non-cleavable and membrane permeable crosslinker that can be used to create sulfhydryl-reactive, maleimide-activated agents (e.g., polypeptides, antibodies) for subsequent reaction with p97 polypeptide sequences. NHS esters react with primary amines at pH 7-9 to form stable amide bonds. Maleimides react with sulfhydryl groups at pH 6.5-7.5 to form stable thioether bonds. Thus, the amine reactive NHS ester of SMCC crosslinks rapidly with primary amines of an agent and the resulting sulfhydryl-reactive maleimide group is then available to react with cysteine residues of p97 to yield specific conjugates of interest.

In certain specific embodiments, the p97 polypeptide sequence is modified to contain exposed sulfhydryl groups to facilitate crosslinking, e.g., to facilitate crosslinking to a maleimide-activated agent. In a more specific embodiment, the p97 polypeptide sequence is modified with a reagent which modifies primary amines to add protected thiol sulfhydryl groups. In an even more specific embodiment, the reagent N-succinimidyl-S-acetylthioacetate (SATA) (Thermo Scientific) is used to produce thiolated p97 polypeptides.

In other specific embodiments, a maleimide-activated agent is reacted under suitable conditions with thiolated p97 polypeptides to produce a conjugate of the present invention. It will be understood that by manipulating the ratios of SMCC, SATA, agent, and p97 polypeptide in these reactions it is possible to produce conjugates having differing stoichiometries, molecular weights and properties.

In still other illustrative embodiments, conjugates are made using bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particular coupling agents include N-succinimidyl-3-(2-pyridyldithio)propionate (SPDP) (Carlsson et al., Biochem. J. 173:723-737 [1978]) and N-succinimidyl-4-(2-pyridylthio)pentanoate (SPP) to provide for a disulfide linkage.

The specific crosslinking strategies discussed herein are but a few of many examples of suitable conjugation strategies that may be employed in producing conjugates of the invention. It will be evident to those skilled in the art that a variety of other bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Particular embodiments may employ one or more aldehyde tags to facilitate conjugation between a p97 polypeptide and an agent (see U.S. Pat. Nos. 8,097,701 and 7,985,783, incorporated by reference). Here, enzymatic modification at a sulfatase motif of the aldehyde tag through action of a formylglycine generating enzyme (FGE) generates a formylglycine (FGly) residue. The aldehyde moiety of the FGly residue can then be exploited as a chemical handle for site-specific attachment of a moiety of interest to the polypeptide. In some aspects, the moiety of interest is a small molecule, peptoid, aptamer, or peptide mimetic. In some aspects, the moiety of interest is another polypeptide, such as an antibody.

Polypeptides with the above-described motif can be modified by an FGE enzyme to generate a motif having a FGly residue, which, as noted above, can then be used for site-specific attachment of an agent, such as a second polypeptide, for instance, via a linker moiety. Such modifications can be performed, for example, by expressing the sulfatase motif-containing polypeptide (e.g., p97, antibody) in a mammalian, yeast, or bacterial cell that expresses an FGE enzyme or by in vitro modification of isolated polypeptide with an isolated FGE enzyme (see Wu et al., PNAS. 106:3000-3005, 2009; Rush and Bertozzi, J. Am Chem Soc. 130:12240-1, 2008; and Carlson et al., J Biol Chem. 283:20117-25, 2008).

The agent or non-aldehyde tag-containing polypeptide (e.g., antibody, p97 polypeptide) can be functionalized with one or more aldehyde reactive groups such as aminooxy, hydrazide, and thiosemicarbazide, and then covalently linked to the aldehyde tag-containing polypeptide via the at least one FGly residue, to form an aldehyde reactive linkage. The attachment of an aminooxy functionalized agent (or non-aldehyde tag-containing polypeptide) creates an oxime linkage between the FGly residue and the functionalized agent (or non-aldehyde tag-containing polypeptide); attachment of a hydrazide-functionalized agent (or non-aldehyde tag-containing polypeptide) creates a hydrazine linkage between the FGly residue and the functionalized agent (or non-aldehyde tag-containing polypeptide); and attachment of a thiosemicarbazide-functionalized agent (or non-aldehyde tag-containing polypeptide) creates a hydrazine carbothiamide linkage between the FGly residue and the functionalized agent (or non-aldehyde tag-containing polypeptide). Hence, in these and related embodiments, R1 can be a linkage that comprises a Schiff base, such as an oxime linkage, a hydrazine linkage, or a hydrazine carbothiamide linkage.

Certain embodiments include conjugates of (i) a sulfatase motif (or aldehyde tag)-containing p97 polypeptide and (ii) a sulfatase motif (or aldehyde tag)-containing polypeptide agent (A), where (i) and (ii) are covalently linked via their respective FGly residues, optionally via a bi-functionalized linker moiety or group.

In some embodiments, the aldehyde tag-containing p97 polypeptide and the aldehyde tag-containing agent are linked (e.g., covalently linked) via a multi-functionalized linker (e.g., bi-functionalized linker), the latter being functionalized with the same or different aldehyde reactive group(s). In these and related embodiments, the aldehyde reactive groups allow the linker to form a covalent bridge between the p97 polypeptide and the agent via their respective FGly residues. Linker moieties include any moiety or chemical that can be functionalized and preferably bi- or multi-functionalized with one or more aldehyde reactive groups. Particular examples include peptides, water-soluble polymers, detectable entities, other therapeutic compounds (e.g., cytotoxic compounds), biotin/streptavidin moieties, and glycans (see Hudak et al., J Am Chem Soc. 133:16127-35, 2011).

Specific examples of glycans (or glycosides) include aminooxy glycans, such as higher-order glycans composed of glycosyl N-pentenoyl hydroxamates intermediates (supra). Exemplary linkers are described herein, and can be functionalized with aldehyde reactive groups according to routine techniques in the art (see, e.g., Carrico et al., Nat Chem Biol. 3:321-322, 2007; and U.S. Pat. Nos. 8,097,701 and 7,985,783).

p97 conjugates can also be prepared by a various “click chemistry” techniques, including reactions that are modular, wide in scope, give very high yields, generate mainly inoffensive byproducts that can be removed by non-chromatographic methods, and can be stereospecific but not necessarily enantioselective (see Kolb et al., Angew Chem Int Ed Engl. 40:2004-2021, 2001). Particular examples include conjugation techniques that employ the Huisgen 1,3-dipolar cycloaddition of azides and alkynes, also referred to as “azide-alkyne cycloaddition” reactions (see Hein et al., Pharm Res. 25:2216-2230, 2008). Non-limiting examples of azide-alkyne cycloaddition reactions include copper-catalyzed azide-alkyne cycloaddition (CuAAC) reactions and ruthenium-catalyzed azide-alkyne cycloaddition (RuAAC) reactions.

CuAAC works over a broad temperature range, is insensitive to aqueous conditions and a pH range over 4 to 12, and tolerates a broad range of functional groups (see Himo et al, J Am Chem Soc. 127:210-216, 2005). The active Cu(I) catalyst can be generated, for example, from Cu(I) salts or Cu(II) salts using sodium ascorbate as the reducing agent. This reaction forms 1,4-substituted products, making it region-specific (see Hein et al., supra).

RuAAC utilizes pentamethylcyclopentadienyl ruthenium chloride [Cp*RuCl] complexes that are able to catalyze the cycloaddition of azides to terminal alkynes, regioselectively leading to 1,5-disubstituted 1,2,3-triazoles (see Rasmussen et al., Org. Lett. 9:5337-5339, 2007). Further, and in contrast to CuAAC, RuAAC can also be used with internal alkynes to provide fully substituted 1,2,3-triazoles.

Certain embodiments thus include p97 polypeptides that comprise at least one unnatural amino acid with an azide side-chain or an alkyne side-chain, including internal and terminal unnatural amino acids (e.g., N-terminal, (-terminal). Certain of these p97 polypeptides can be formed by in vivo or in vitro (e.g., cell-free systems) incorporation of unnatural amino acids that contain azide side-chains or alkyne side-chains. Exemplary in vivo techniques include cell culture techniques, for instance, using modified E. coli (see Travis and Schultz, The Journal of Biological Chemistry. 285:11039-44, 2010; and Deiters and Schultz, Bioorganic & Medicinal Chemistry Letters. 15:1521-1524, 2005), and exemplary in vitro techniques include cell-free systems (see Bundy, Bioconjug Chem. 21:255-63, 2010).

In some embodiments, a p97 polypeptide that comprises at least one unnatural amino acid with an azide side-chain is conjugated by azide-alkyne cycloaddition to an agent (or linker) that comprises at least one alkyne group, such as a polypeptide agent that comprises at least one unnatural amino acid with an alkyne side-chain. In other embodiments, a p97 polypeptide that comprises at least one unnatural amino acid with an alkyne side-chain is conjugated by azide-alkyne cycloaddition to an agent (or linker) that comprises at least one azide group, such as a polypeptide agent that comprises at least one unnatural amino acid with an azide side-chain. Hence, certain embodiments include conjugates that comprise a p97 polypeptide covalently linked to an agent via a 1,2,3-triazole linkage.

In certain embodiments, the unnatural amino acid with the azide side-chain and/or the unnatural amino acid with alkyne side-chain are terminal amino acids (N-terminal, (-terminal). In certain embodiments, one or more of the unnatural amino acids are internal.

For instance, certain embodiments include a p97 polypeptide that comprises an N-terminal unnatural amino acid with an azide side-chain conjugated to an agent that comprises an alkyne group. Some embodiments, include a p97 polypeptide that comprises a (-terminal unnatural amino acid with an azide side-chain conjugated to an agent that comprises an alkyne group. Particular embodiments include a p97 polypeptide that comprises an N-terminal unnatural amino acid with an alkyne side-chain conjugated to an agent that comprises an azide side-group. Further embodiments include a p97 polypeptide that comprises an (-terminal unnatural amino acid with an alkyne side-chain conjugated to an agent that comprises an azide side-group. Some embodiments include a p97 polypeptide that comprises at least one internal unnatural amino acid with an azide side-chain conjugated to an agent that comprises an alkyne group. Additional embodiments include a p97 polypeptide that comprises at least one internal unnatural amino acid with an alkyne side-chain conjugated to an agent that comprises an azide side-group.

Particular embodiments include a p97 polypeptide that comprises an N-terminal unnatural amino acid with an azide side-chain conjugated to a polypeptide agent that comprises an N-terminal unnatural amino acid with an alkyne side-chain. Other embodiments include a p97 polypeptide that comprises a (-terminal unnatural amino acid with an azide side-chain conjugated to a polypeptide agent that comprises a (-terminal unnatural amino acid with an alkyne side-chain. Still other embodiments include a p97 polypeptide that comprises an N-terminal unnatural amino acid with an azide side-chain conjugated to a polypeptide agent that comprises a (-terminal unnatural amino acid with an alkyne side-chain. Further embodiments include a p97 polypeptide that comprises a (-terminal unnatural amino acid with an azide side-chain conjugated to a polypeptide agent that comprises an N-terminal unnatural amino acid with an alkyne side-chain.

Other embodiments include a p97 polypeptide that comprises an N-terminal unnatural amino acid with an alkyne side-chain conjugated to a polypeptide agent that comprises an N-terminal unnatural amino acid with an azide side-chain. Still further embodiments include a p97 polypeptide that comprises a (-terminal unnatural amino acid with an alkyne side-chain conjugated to a polypeptide agent that comprises a (-terminal unnatural amino acid with an azide side-chain. Additional embodiments include a p97 polypeptide that comprises an N-terminal unnatural amino acid with an alkyne side-chain conjugated to a polypeptide agent that comprises a (-terminal unnatural amino acid with an azide side-chain. Still further embodiments include a p97 polypeptide that comprises a (-terminal unnatural amino acid with an alkyne side-chain conjugated to a polypeptide agent that comprises an N-terminal unnatural amino acid with an azide side-chain.

Also included are methods of producing a p97 conjugate, comprising: (a) performing an azide-alkyne cycloaddition reaction between (i) a p97 polypeptide that comprises at least one unnatural amino acid with an azide side-chain and an agent that comprises at least one alkyne group (for instance, an unnatural amino acid with an alkyne side chain); or (ii) a p97 polypeptide that comprises at least one unnatural amino acid with an alkyne side-chain and an agent that comprises at least one azide group (for instance, an unnatural amino acid with an azide side-chain); and (b) isolating a p97 conjugate from the reaction, thereby producing a p97 conjugate.

In the case where the p97 conjugate is a fusion polypeptide, the fusion polypeptide may generally be prepared using standard techniques. Preferably, however, a fusion polypeptide is expressed as a recombinant polypeptide in an expression system, described herein and known in the art. Fusion polypeptides of the invention can contain one or multiple copies of a p97 polypeptide sequence and may contain one or multiple copies of a polypeptide-based agent of interest (e.g., antibody or antigen-binding fragment thereof), present in any desired arrangement.

For fusion proteins, DNA sequences encoding the p97 polypeptide, the polypeptide agent (e.g., antibody), and optionally peptide linker components may be assembled separately, and then ligated into an appropriate expression vector. The 3′ end of the DNA sequence encoding one polypeptide component is ligated, with or without a peptide linker, to the 5′ end of a DNA sequence encoding the other polypeptide component(s) so that the reading frames of the sequences are in phase. The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the most (-terminal polypeptide. This permits translation into a single fusion polypeptide that retains the biological activity of both component polypeptides.

Similar techniques, mainly the arrangement of regulatory elements such as promoters, stop codons, and transcription termination signals, can be applied to the recombinant production of non-fusion proteins, for instance, p97 polypeptides and polypeptide agents (e.g., antibody agents) for the production of non-fusion conjugates.

Polynucleotides and fusion polynucleotides of the invention can contain one or multiple copies of a nucleic acid encoding a p97 polypeptide sequence, and/or may contain one or multiple copies of a nucleic acid encoding a polypeptide agent.

In some embodiments, a nucleic acids encoding a subject p97 polypeptide, polypeptide agent, and/or p97-polypeptide fusion are introduced directly into a host cell, and the cell incubated under conditions sufficient to induce expression of the encoded polypeptide(s). The polypeptide sequences of this disclosure may be prepared using standard techniques well known to those of skill in the art in combination with the polypeptide and nucleic acid sequences provided herein.

Therefore, according to certain related embodiments, there is provided a recombinant host cell which comprises a polynucleotide or a fusion polynucleotide that encodes a polypeptide described herein. Expression of a p97 polypeptide, polypeptide agent, or p97-polypeptide agent fusion in the host cell may conveniently be achieved by culturing under appropriate conditions recombinant host cells containing the polynucleotide. Following production by expression, the polypeptide(s) may be isolated and/or purified using any suitable technique, and then used as desired.

Systems for cloning and expression of a polypeptide in a variety of different host cells are well known. Suitable host cells include bacteria, mammalian cells, yeast and baculovirus systems.

Mammalian cell lines available in the art for expression of a heterologous polypeptide include Chinese hamster ovary (CHO) cells, Hela cells, baby hamster kidney cells, HEK-293 cells, NSO mouse melanoma cells and many others. A common, preferred bacterial host is E. coli. The expression of polypeptides in prokaryotic cells such as E. coli is well established in the art. For a review, see for example Pluckthun, A. Bio/Technology. 9:545-551 (1991). Expression in eukaryotic cells in culture is also available to those skilled in the art as an option for recombinant production of polypeptides (see Ref, Curr. Opinion Biotech. 4:573-576, 1993; and Trill et al., Curr. Opinion Biotech. 6:553-560, 1995.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992, or subsequent updates thereto.

The term “host cell” is used to refer to a cell into which has been introduced, or which is capable of having introduced into it, a nucleic acid sequence encoding one or more of the polypeptides described herein, and which further expresses or is capable of expressing a selected gene of interest, such as a gene encoding any herein described polypeptide. The term includes the progeny of the parent cell, whether or not the progeny are identical in morphology or in genetic make-up to the original parent, so long as the selected gene is present. Host cells may be chosen for certain characteristics, for instance, the expression of a formylglycine generating enzyme (FGE) to convert a cysteine or serine residue within a sulfatase motif into a formylglycine (FGly) residue, or the expression of aminoacyl tRNA synthetase(s) that can incorporate unnatural amino acids into the polypeptide, including unnatural amino acids with an azide side-chain, alkyne side-chain, or other desired side-chain, to facilitate conjugation.

Accordingly there is also contemplated a method comprising introducing such nucleic acid(s) into a host cell. The introduction of nucleic acids may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g., by culturing host cells under conditions for expression of the gene. In one embodiment, the nucleic acid is integrated into the genome (e.g. chromosome) of the host cell. Integration may be promoted by inclusion of sequences which promote recombination with the genome, in accordance-with standard techniques.

The present invention also provides, in certain embodiments, a method which comprises using a nucleic acid construct described herein in an expression system in order to express a particular polypeptide, such as a p97 polypeptide, polypeptide agent, or p97-polypeptide agent fusion protein as described herein.

As noted above, certain p97 conjugates, such as fusion proteins, may employ one or more linker groups, including non-peptide linkers (e.g., non-proteinaceous linkers) and peptide linkers. Such linkers can be stable linkers or releasable linkers.

Exemplary non-peptide stable linkages include succinimide, propionic acid, carboxymethylate linkages, ethers, carbamates, amides, amines, carbamides, imides, aliphatic C—C bonds, thio ether linkages, thiocarbamates, thiocarbamides, and the like. Generally, a hydrolytically stable linkage is one that exhibits a rate of hydrolysis of less than about 1-2% to 5% per day under physiological conditions.

Exemplary non-peptide releasable linkages include carboxylate ester, phosphate ester, anhydride, acetal, ketal, acyloxyalkyl ether, imine, orthoester, thio ester, thiol ester, carbonate, and hydrazone linkages. Other illustrative examples of releasable linkers can be benzyl elimination-based linkers, trialkyl lock-based linkers (or trialkyl lock lactonization based), bicine-based linkers, and acid labile linkers. Among the acid labile linkers can be disulfide bond, hydrazone-containing linkers and thiopropionate-containing linkers. Also included are linkers that are releasable or cleavable during or upon internalization into a cell. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.). In one embodiment, an acid-labile linker may be used (Cancer Research 52:127-131, 1992; and U.S. Pat. No. 5,208,020). Further details are known to those skilled in the art. See, For example, U.S. Pat. No. 9,364,567.

In certain embodiments, “water soluble polymers” are used in a linker for coupling a p97 polypeptide sequence to an agent of interest. A “water-soluble polymer” refers to a polymer that is soluble in water and is usually substantially non-immunogenic, and usually has an atomic molecular weight greater than about 1,000 Daltons. Attachment of two polypeptides via a water-soluble polymer can be desirable as such modification(s) can increase the therapeutic index by increasing serum half-life, for instance, by increasing proteolytic stability and/or decreasing renal clearance. Additionally, attachment via of one or more polymers can reduce the immunogenicity of protein pharmaceuticals.

Particular examples of water soluble polymers include polyethylene glycol, polypropylene glycol, polyoxyalkylenes, or copolymers of polyethylene glycol, polypropylene glycol, and the like.

In some embodiments, the water-soluble polymer has an effective hydrodynamic molecular weight of greater than about 10,000 Da, greater than about 20,000 to 500,000 Da, greater than about 40,000 Da to 300,000 Da, greater than about 50,000 Da to 70,000 Da, usually greater than about 60,000 Da. The “effective hydrodynamic molecular weight” refers to the effective water-solvated size of a polymer chain as determined by aqueous-based size exclusion chromatography (SEC). When the water-soluble polymer contains polymer chains having polyalkylene oxide repeat units, such as ethylene oxide repeat units, each chain can have an atomic molecular weight of between about 200 Da and about 80,000 Da, or between about 1,500 Da and about 42,000 Da, with 2,000 to about 20,000 Da being of particular interest. Linear, branched, and terminally charged water soluble polymers are also included.

Polymers useful as linkers between aldehyde tagged polypeptides can have a wide range of molecular weights, and polymer subunits. These subunits may include a biological polymer, a synthetic polymer, or a combination thereof. Examples of such water-soluble polymers include: dextran and dextran derivatives, including dextran sulfate, P-amino cross linked dextrin, and carboxymethyl dextrin, cellulose and cellulose derivatives, including methylcellulose and carboxymethyl cellulose, starch and dextrines, and derivatives and hydroylactes of starch, polyalklyene glycol and derivatives thereof, including polyethylene glycol (PEG), methoxypolyethylene glycol, polyethylene glycol homopolymers, polypropylene glycol homopolymers, copolymers of ethylene glycol with propylene glycol, wherein said homopolymers and copolymers are unsubstituted or substituted at one end with an alkyl group, heparin and fragments of heparin, polyvinyl alcohol and polyvinyl ethyl ethers, polyvinylpyrrolidone, aspartamide, and polyoxyethylated polyols, with the dextran and dextran derivatives, dextrine and dextrine derivatives. It will be appreciated that various derivatives of the specifically described water-soluble polymers are also included.

Water-soluble polymers are known in the art, particularly the polyalkylene oxide-based polymers such as polyethylene glycol “PEG” (see Poly(ethylene glycol) Chemistry: Biotechnical and Biomedical Applications, J. M. Harris, Ed., Plenum Press, New York, N.Y. (1992); and Poly(ethylene glycol) Chemistry and Biological Applications, J. M. Harris and S. Zalipsky, Eds., ACS (1997); and International Patent Applications: WO 90/13540, WO 92/00748, WO 92/16555, WO 94/04193, WO 94/14758, WO 94/17039, WO 94/18247, WO 94/28937, WO 95/11924, WO 96/00080, WO 96/23794, WO 98/07713, WO 98/41562, WO 98/48837, WO 99/30727, WO 99/32134, WO 99/33483, WO 99/53951, WO 01/26692, WO 95/13312, WO 96/21469, WO 97/03106, WO 99/45964, and U.S. Pat. Nos. 4,179,337; 5,075,046; 5,089,261; 5,100,992; 5,134,192; 5,166,309; 5,171,264; 5,213,891; 5,219,564; 5,275,838; 5,281,698; 5,298,643; 5,312,808; 5,321,095; 5,324,844; 5,349,001; 5,352,756; 5,405,877; 5,455,027; 5,446,090; 5,470,829; 5,478,805; 5,567,422; 5,605,976; 5,612,460; 5,614,549; 5,618,528; 5,672,662; 5,637,749; 5,643,575; 5,650,388; 5,681,567; 5,686,110; 5,730,990; 5,739,208; 5,756,593; 5,808,096; 5,824,778; 5,824,784; 5,840,900; 5,874,500; 5,880,131; 5,900,461; 5,902,588; 5,919,442; 5,919,455; 5,932,462; 5,965,119; 5,965,566; 5,985,263; 5,990,237; 6,011,042; 6,013,283; 6,077,939; 6,113,906; 6,127,355; 6,177,087; 6,180,095; 6,194,580; 6,214,966, incorporated by reference).

Exemplary polymers of interest include those containing a polyalkylene oxide, polyamide alkylene oxide, or derivatives thereof, including polyalkylene oxide and polyamide alkylene oxide comprising an ethylene oxide repeat unit. Further exemplary polymers of interest include a polyamide having a molecular weight greater than about 1,000 Daltons. Further exemplary water-soluble repeat units comprise an ethylene oxide. The number of such water-soluble repeat units can vary significantly, with the usual number of such units being from 2 to 500, 2 to 400, 2 to 300, 2 to 200, 2 to 100, and most usually 2 to 50.

In certain embodiments, a peptide linker sequence may be employed to separate or couple the components of a p97 conjugate. For instance, for polypeptide-polypeptide conjugates, peptide linkers can separate the components by a distance sufficient to ensure that each polypeptide folds into its secondary and tertiary structures. Such a peptide linker sequence may be incorporated into the conjugate (e.g., fusion protein) using standard techniques described herein and well-known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180.

In certain illustrative embodiments, a peptide linker is between about 1 to 5 amino acids, between 5 to 10 amino acids, between 5 to 25 amino acids, between 5 to 50 amino acids, between 10 to 25 amino acids, between 10 to 50 amino acids, between 10 to 100 amino acids, or any intervening range of amino acids. In other illustrative embodiments, a peptide linker comprises about 1, 5, 10, 15, 20, 25, 30, 35, 40, 45, 50 or more amino acids in length. Particular linkers can have an overall amino acid length of about 1-200 amino acids, 1-150 amino acids, 1-100 amino acids, 1-90 amino acids, 1-80 amino acids, 1-70 amino acids, 1-60 amino acids, 1-50 amino acids, 1-40 amino acids, 1-30 amino acids, 1-20 amino acids, 1-10 amino acids, 1-5 amino acids, 1-4 amino acids, 1-3 amino acids, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more amino acids.

A peptide linker may employ any one or more naturally-occurring amino acids, non-naturally occurring amino acid(s), amino acid analogs, and/or amino acid mimetics as described elsewhere herein and known in the art. Certain amino acid sequences which may be usefully employed as linkers include those disclosed in Maratea et al., Gene 40:39-46, 1985; Murphy et al., PNAS USA. 83:8258-8262, 1986;

U.S. Pat. Nos. 4,935,233 and 4,751,180. Particular peptide linker sequences contain Gly, Ser, and/or Asn residues. Other near neutral amino acids, such as Thr and Ala may also be employed in the peptide linker sequence, if desired. Other combinations of these and related amino acids will be apparent to persons skilled in the art.

In specific embodiments, the linker sequence comprises a Gly3 linker sequence, which includes three glycine residues. In particular embodiments, flexible linkers can be rationally designed using a computer program capable of modeling both DNA-binding sites and the peptides themselves (Desjarlais & Berg, PNAS. 90:2256-2260, 1993; and PNAS. 91:11099-11103, 1994) or by phage display methods.

The peptide linkers may be physiologically stable or may include a releasable linker such as a physiologically degradable or enzymatically degradable linker (e.g., proteolytically cleavable linker). In certain embodiments, one or more releasable linkers can result in a shorter half-life and more rapid clearance of the conjugate. These and related embodiments can be used, for example, to enhance the solubility and blood circulation lifetime of p97 conjugates in the bloodstream, while also delivering an agent into the bloodstream (or across the BBB) that, subsequent to linker degradation, is substantially free of the p97 sequence. These aspects are especially useful in those cases where polypeptides or other agents, when permanently conjugated to a p97 sequence, demonstrate reduced activity. By using the linkers as provided herein, such antibodies can maintain their therapeutic activity when in conjugated form. In these and other ways, the properties of the p97 conjugates can be more effectively tailored to balance the bioactivity and circulating half-life of the antibodies over time.

Enzymatically degradable linkages suitable for use in particular embodiments of the present invention include, but are not limited to: an amino acid sequence cleaved by a serine protease such as thrombin, chymotrypsin, trypsin, elastase, kallikrein, or substilisin.

Enzymatically degradable linkages suitable for use in particular embodiments of the present invention also include amino acid sequences that can be cleaved by a matrix metalloproteinase such as collagenase, stromelysin, and gelatinase.

Enzymatically degradable linkages suitable for use in particular embodiments of the present invention also include amino acid sequences that can be cleaved by an angiotensin converting enzyme.

Enzymatically degradable linkages suitable for use in particular embodiments of the present invention also include amino acid sequences that can be degraded by cathepsin B.

In certain embodiments, however, any one or more of the non-peptide or peptide linkers are optional. For instance, linker sequences may not required in a fusion protein where the first and second polypeptides have non-essential N-terminal and/or (-terminal amino acid regions that can be used to separate the functional domains and prevent steric interference.

The functional properties of the p97 polypeptides and p97 polypeptide conjugates described herein may be assessed using a variety of methods known to the skilled person, including, e.g., affinity/binding assays (for example, surface plasmon resonance, competitive inhibition assays); cytotoxicity assays, cell viability assays, cell proliferation or differentiation assays, cancer cell and/or tumor growth inhibition using in vitro or in vivo models. For instance, the conjugates described herein may be tested for effects on receptor internalization, in vitro and in vivo efficacy, etc., including the rate of transport across the blood brain barrier. Such assays may be performed using well-established protocols known to the skilled person (see e.g., Current Protocols in Molecular Biology (Greene Publ. Assoc. Inc. & John Wiley & Sons, Inc., NY, NY); Current Protocols in Immunology (Edited by: John E. Coligan, Ada M. Kruisbeek, David H. Margulies, Ethan M. Shevach, Warren Strober 2001 John Wiley & Sons, NY, NY); or commercially available kits.

Methods of Use and Pharmaceutical Compositions

Certain embodiments of the present invention relate to methods of using the compositions of p97 polypeptides and p97 conjugates described herein. Examples of such methods include methods of treatment and methods of diagnosis, including for instance, the use of p97 conjugates for the treatment of Lewy body dementias (dementia of Lewy bodies and Parkinson's disease dementia). Combination therapy including the administration of the p97 conjugates of the invention with other therapies for treating Lewy body dementias may be employed.

Accordingly, certain embodiments include methods of treating a subject in need thereof, comprising administering a composition that comprises a p97 conjugate described herein. Also included are methods of delivering an agent to the nervous system (e.g., central nervous system tissues) of a subject, comprising administering a composition that comprises a p97 conjugate described herein. In certain of these and related embodiments, the methods increase the rate of delivery of the agent to the central nervous system tissues, relative, for example, to delivery by a composition that comprises the agent alone.

In some instances, a subject has a disease, disorder, or condition of the CNS, where increased delivery of a therapeutic agent across the blood brain barrier to CNS tissues relative to peripheral tissues can improve treatment, for instance, by reducing side-effects associated with exposure of an agent to peripheral tissues. Exemplary diseases, disorders, and conditions of the CNS include lysosomal storage diseases such as Gaucher disease.

In some instances, the subject has or is at risk for having one or more lysosomal storage diseases. Certain methods thus relate to the treatment of lysosomal storage diseases in a subject in need thereof, optionally those lysosomal storage diseases associated with the central nervous system. Exemplary lysosomal storage diseases include aspartylglucosaminuria, cholesterol ester storage disease, Wolman disease, cystinosis, Danon disease, Fabry disease, Farber lipogranulomatosis, Farber disease, fucosidosis, galactosialidosis types 1/11, Gaucher disease types 1/11/111, Gaucher disease, globoid cell leucodystrophy, Krabbe disease, glycogen storage disease II, Pompe disease, GMI-gangliosidosis types 1/11/111, GM2-gangliosidosis type I, Tay Sachs disease, GM2-gangliosidosis type II, Sandhoff disease, GM2-gangliosidosis, a-mannosidosis types 1/11, -mannosidosis, metachromatic leucodystrophy, mucolipidosis type I, sialidosis types 1/11 mucolipidosis types 11/1111-cell disease, mucolipidosis type IIIC pseudo-Hurler polydystrophy, mucopolysaccharidosis type I, mucopolysaccharidosis type II, Hunter syndrome, mucopolysaccharidosis type IIIA, Sanfilippo syndrome, mucopolysaccharidosis type IIIB, mucopolysaccharidosis type IIIC, mucopolysaccharidosis type 111 D, mucopolysaccharidosis type IVA, Morquio syndrome, mucopolysaccharidosis type IVB Morquio syndrome, mucopolysaccharidosis type VI, mucopolysaccharidosis type VII, Sly syndrome, mucopolysaccharidosis type IX, multiple sulfatase deficiency, neuronal ceroid lipofuscinosis, CLNI Batten disease, Niemann-Pick disease types NB, Niemann-Pick disease, Niemann-Pick disease type CI, Niemann-Pick disease type C2, pycnodysostosis, Schindler disease types 1/11, Schindler disease, and sialic acid storage disease. In these and related embodiments, the p97 polypeptide can be conjugated to one or more polypeptides associated with a lysosomal storage disease, as described herein.

In some instances, a subject has a disease, disorder, or condition of the CNS, where increased delivery of a therapeutic agent across the blood brain barrier to CNS tissues relative to peripheral tissues can improve treatment, for instance, by increasing or restoring the GCase activity or decrease the accumulation of α-synuclein. Exemplary diseases, disorders, and conditions of the CNS include Lewy body dementias (dementia of Lewy bodies and Parkinson's disease dementia and Parkinson's disease) and synucleinopathies (Parkinson's disease, dementia of Lewy bodies and multiple system atrophy).

In some instances, the subject has or is at risk for having one or more GBA1 gene mutation associated diseases. Certain methods thus relate to the treatment of GBA1 gene mutation associated diseases in a subject in need thereof, optionally those GBA1 gene mutation associated diseases affect the central nervous system. Exemplary GBA1 gene mutation associated diseases include Gaucher diseases, Gaucher disease type I/II/III, Parkinson's disease, parkinson's disease dementia, dementia of Lewy bodies, Lewy body dementia(s), multiple system atrophy, REM sleep behavior disorders.

Lewy body disease has been indicated as the second most common neurodegenerative dementia behind Alzheimer disease, and make up between 15 to 30% of all neurodegenerative dementias. See, McKeith I G, Boeve B F, Dickson D W, et al. Diagnosis and management of dementia with Lewy bodies: fourth consensus report of the DLB consortium. Neurology. 2017; 89(1):88-100; and Walker Z, Possin K L, Boeve B F, Aarsland D. Lewy body dementias, Lancet. 2015; 386(10004):1683-1697. GBA1 mutations are a common risk factor for PD and dementia of Lewy bodies patients, which shares many similar genetic risk factors, prodromal features and neuropathological features at autopsy. See, Mov Disord. 2020 January; 35(1):5-19. doi: 10.1002/mds.27867. Epub 2019 Oct. 29, Pathological Influences on Clinical Heterogeneity in Lewy Body Diseases, Coughlin D G1,2,3, Hurtig H I1, Irwin D J. Clinically, patients with heterozygous mutation of GBA1 gene may be indistinguishable from idiopathic PD patients, however, they are more prevalent to cognitive impairment and earlier onset age. See, Clark L N, Ross B M, Wang Y et al (2007) Mutations in the glucocerebrosidase gene are associated with early-onset Parkinson disease. Neurology 69:1270-1277; and 16. Clark L N, Kartsaklis L A, Wolf Gilbert R et al (2009) Association of glucocerebrosidase mutations with dementia with lewy bodies, Arch Neurol 66:578-583. Significant decrease was found in the postmortem brain tissue of PD brains of heterozygote GBA mutation in the substantia nigra (decrease of 58% GCase activity), wile 33% decrease activity was found in the substantia nigra of sporadic PD brains. See, Gegg M E, Burke D, Heales S J R, Cooper J M, Hardy J, Wood N W & Schapira A H V (2012) Glucocerebrosidase deficiency in substantia nigra of Parkinson disease brains. Ann Neurol 72, 455-463. The risk of developing DLB is about three times greater than developing PD for GBA1 carriers, with earlier age of onset and higher disease severity. See, Riboldi G M, Di Fonzo A B. GBA, Gaucher Disease, and Parkinson's Disease: From Genetic to Clinic to New Therapeutic Approaches. Cells. 2019; 8(4):364. Published 2019 Apr. 19. doi:10.3390/cells8040364. Neuropathological data have shown reduced GBA1 expression in both specific regions of brain and in the peripheral blood for DLB and PD patients. See, Perez-Roca, L.; Adame-Castillo, C.; Campdelacreu, J.; Ispierto, L.; Vilas, D.; Rene, R.; Alvarez, R., Gascon-Bayarri, J.; Serrano-Munoz, M. A.; Ariza, A.; et al. Glucocerebrosidase mRNA is Diminished in Brain of Lewy Body Diseases and Changes with Disease Progression in Blood. Aging Dis. 2018, 9, 208-219.

Methods for identifying subjects with one or more of the diseases or conditions described herein are known in the art.

Also included are methods for imaging an organ or tissue component in a subject, comprising (a) administering to the subject a composition comprising a human p97 (melanotransferrin) polypeptide, or a variant thereof, where the p97 polypeptide is conjugated to a detectable entity, and (b) visualizing the detectable entity in the subject, organ, or tissue.

In particular embodiments, the organ or tissue compartment comprises the central nervous system (e.g., brain, brainstem, spinal cord). In specific embodiments, the organ or tissue compartment comprises the brain or a portion thereof, for instance, the parenchyma of the brain.

A variety of methods can be employed to visualize the detectable entity in the subject, organ, or tissue. Exemplary non-invasive methods include radiography, such as fluoroscopy and projectional radiographs, CT-scanning or CAT-scanning (computed tomography (CT) or computed axial tomography (CAT)), whether employing X-ray CT-scanning, positron emission tomography (PET), or single photon emission computed tomography (SPECT), and certain types of magnetic resonance imaging (MRI), especially those that utilize contrast agents, including combinations thereof. Merely by way of example, PET can be performed with positron-emitting contrast agents or radioisotopes such as 18 F, SPECT can be performed with gamma-emitting contrast agents or radioisotopes and MRI can be performed with contrast agents or radioisotopes. Any one or more of these exemplary contrast agents or radioisotopes can be conjugated to or otherwise incorporated into a p97 polypeptide and administered to a subject for imaging purposes.

For instance, p97 polypeptides can be directly labeled with one or more of these radioisotopes, or conjugated to molecules (e.g., small molecules) that comprise one or more of these radioisotopic contrast agents, or any others described herein.

For in vivo use, for instance, for the treatment of human disease, medical imaging, or testing, the conjugates described herein are generally incorporated into a pharmaceutical composition prior to administration. A pharmaceutical composition comprises one or more of the p97 polypeptides or conjugates described herein in combination with a physiologically acceptable carrier or excipient.

To prepare a pharmaceutical composition, an effective or desired amount of one or more of the p97 polypeptides or conjugates is mixed with any pharmaceutical carrier(s) or excipient known to those skilled in the art to be suitable for the particular mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously, suitable carriers include physiological saline or phosphate buffered saline (PBS), and solutions containing thickening and solubilizing agents, such as glucose, polyethylene glycol, polypropylene glycol and mixtures thereof.

Administration of the polypeptides and conjugates described herein, in pure form or in an appropriate pharmaceutical composition, can be carried out via any of the accepted modes of administration of agents for serving similar utilities. The pharmaceutical compositions can be prepared by combining a polypeptide or conjugate or conjugate-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other anti-cancer agents as described elsewhere herein) and/or suitable excipients such as salts, buffers and stabilizers may, but need not, be present within the composition.

Administration may be achieved by a variety of different routes, including oral, parenteral, nasal, intravenous, intradermal, subcutaneous or topical. Preferred modes of administration depend upon the nature of the condition to be treated or prevented.

Carriers can include, for example, pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In certain aspects, the p97 polypeptide sequence and the agent are each, individually or as a pre-existing conjugate, bound to or encapsulated within a particle, e.g., a nanoparticle, bead, lipid formulation, lipid particle, or liposome, e.g., immunoliposome. For instance, in particular embodiments, the p97 polypeptide sequence is bound to the surface of a particle, and the agent of interest is bound to the surface of the particle and/or encapsulated within the particle. In some of these and related embodiments, the p97 polypeptide and the agent are covalently or operatively linked to each other only via the particle itself (e.g., nanoparticle, liposome), and are not covalently linked to each other in any other way; that is, they are bound individually to the same particle. In other embodiments, the p97 polypeptide and the agent are first covalently or non-covalently conjugated to each other, as described herein (e.g., via a linker molecule), and are then bound to or encapsulated within a particle (e.g., immunoliposome, nanoparticle). In specific embodiments, the particle is a liposome, and the composition comprises one or more p97 polypeptides, one or more agents of interest, and a mixture of lipids to form a liposome (e.g., phospholipids, mixed lipid chains with surfactant properties). In some aspects, the p97 polypeptide and the agent are individually mixed with the lipid/liposome mixture, such that the formation of liposome structures operatively links the p97 polypeptide and the agent without the need for covalent conjugation. In other aspects, the p97 polypeptide and the agent are first covalently or non-covalently conjugated to each other, as described herein, and then mixed with lipids to form a liposome. The p97 polypeptide, the agent, or the p97-agent conjugate may be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents, such as cytotoxic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described conjugate in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a p97 polypeptide, agent, or conjugate described herein, for treatment of a disease or condition of interest.

A pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of a p97 polypeptide or conjugate as herein disclosed such that a suitable dosage will be obtained. Typically, this amount is at least 0.01% of the agent of interest in the composition. When intended for oral administration, this amount may be varied to be between 0.1 and about 70% of the weight of the composition. Certain oral pharmaceutical compositions contain between about 4% and about 75% of the agent of interest. In certain embodiments, pharmaceutical compositions and preparations according to the present invention are prepared so that a parenteral dosage unit contains between 0.01 to 10% by weight of the agent of interest prior to dilution.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter, and polyethylene glycol.

The pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The pharmaceutical composition in solid or liquid form may include an agent that binds to the conjugate or agent and thereby assists in the delivery of the compound. Suitable agents that may act in this capacity include monoclonal or polyclonal antibodies, one or more proteins or a liposome.

The pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s).

Delivery of the aerosol includes the necessary container, activators, valves, subcontainers, and the like, which together may form a kit. One of ordinary skill in the art, without undue experimentation may determine preferred aerosols.

The compositions comprising conjugates as described herein may be prepared with carriers that protect the conjugates against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The pharmaceutical compositions may be prepared by methodology well known in the pharmaceutical art. For example, a pharmaceutical composition intended to be administered by injection can be prepared by combining a composition that comprises a conjugate as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the conjugate so as to facilitate dissolution or homogeneous suspension of the conjugate in the aqueous delivery system.

The compositions may be administered in a therapeutically effective amount, which will vary depending upon a variety of factors including the activity of the specific compound (e.g., conjugate) employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Generally, a therapeutically effective daily dose is (for a 70 kg mammal) from about 0.001 mg/kg (i.e., ˜0.07 mg) to about 100 mg/kg (i.e., ˜7.0 g); preferably a therapeutically effective dose is (for a 70 kg mammal) from about 0.01 mg/kg (i.e., ˜0.7 mg) to about 50 mg/kg (i.e., ˜3.5 g); more preferably a therapeutically effective dose is (for a 70 kg mammal) from about 1 mg/kg (i.e., ˜70 mg) to about 25 mg/kg (i.e., ˜1.75 g).

### EXAMPLES

The following examples are provided for illustrative purposes and are not intended to limit the scope of the claims which follow.

### Example 1—Fushion

A p97 fragment, DSSHAFTLDELR (SEQ ID NO: 2), is genetically fused to the first amino acid of the N-terminal end of the desired mature enzyme through a linker sequence, e.g., (G₄S)₃, (G₄S)₂ or (EA₃K)₃. The DNA plasmid containing the p97 fragment-enzyme sequence is then cloned into mammalian expression vectors, which is then transfected into cells for protein production. The condition media from the transfection production is then harvested and purified through affinity chromatography.

### Example 2—Conjugation

A p97 fragment, DSSHAFTLDELR (SEQ ID NO: 2), is conjugated to the desired enzyme utilizing a conjugation technique, e.g, SoluLink™ bioconjugation method or malemide-thiol interaction method (See, e.g, https://www.trilinkbiotech.com/solulink/ for information and availability of the Solulink bioconjugation products). The SoluLink bioconjugation is performed by modification of p97 fragment with a 4FB crosslinker and modification of enzyme with a HyNic cross linker. The mixing of the two modified biomolecules will result in the formation of a stable, UV-traceable bond formed by the reaction of a HyNic modified enzyme with a 4FB modified p97 fragment. Malemide-thiol conjugation is performed by modification of enzyme with N-(β-maleimidopropyloxy) succinimide ester (BMPS) resulting in malemide-containing enzyme, as well as addition of a cysteine to the c-terminus of the p97 fragment and subsequent thiol modification of the p97 fragment. The maleimide-containing enzyme is then reacted with the thiol-containing the p97 fragment, with the reaction is quenched by cysteine.

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls. The citation of a reference herein should not be construed as an acknowledgement that such reference is prior art to the present invention.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the methods and compositions of the present invention, where the term comprises is used with respect to the recited steps of the methods or components of the compositions, it is also contemplated that the methods and compositions consist essentially of, or consist of, the recited steps or components. Furthermore, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as being composed of the components prior to mixing, because upon mixing certain components can further react or be transformed into additional materials.

Those skilled in the art will recognize, or be able to ascertain using no more than routine experimentation, numerous equivalents to the specific procedures described herein. Such equivalents are considered to be within the scope of this invention and are covered by the following claims.

All percentages and ratios used herein, unless otherwise indicated, are by weight. It is recognized the mass of an object is often referred to as its weight in everyday usage and for most common scientific purposes, but that mass technically refers to the amount of matter of an object, whereas weight refers to the force experienced by an object due to gravity. Also, in common usage the “weight” (mass) of an object is what one determines when one “weighs” (masses) an object on a scale or balance.

1. A method of treating Lewy body dementia comprising administering to a subject in need thereof a therapeutic payload (i.e. composition) comprising an active agent suitable for treating Lewy body dementia coupled with a p97 polypeptide or fragment thereof, wherein said administration promotes the transport of the therapeutic payload across the blood brain barrier of the subject.

2-3. (canceled)

4. A method according to claim 1 wherein the p97 polypeptide comprises up to about 300 amino acids in length, where the polypeptide comprises an amino acid sequence at least 70% identical to DSSHAFTLDELR (SEQ ID NO:13) or any one or more of SEQ ID NOS: 2 to 19.

5. A method according to claim 1 wherein the p97 polypeptide comprises DSSHAFTLDELR (SEQ ID NO:13) or any one or more of SEQ ID NOS: 2 to 19, optionally including adjacent C-terminal and/or N-terminal sequences as defined by SEQ ID NO:1.

6. A method according to claim 1 wherein the p97 polypeptide comprises 2, 3, 4, or 5 amino acids of DSSHAFTLDELR (SEQ ID NO:13) or SEQ ID NOS: 2 to 19, optionally including any intervening sequences as defined by SEQ ID NO:1.

7. A method according to claim 1 wherein the p97 polypeptide comprises one or both of SEQ ID NO:13 and/or 14, optionally including intervening sequences as defined by SEQ ID NO:1.

8. A method according to claim 1 wherein the p97 polypeptide comprises up to about 250 amino acids in length.

9. A method according to claim 1 wherein said active agent is coupled to said p97 polypeptide or fragment thereof with a linker.

10. (canceled)

11. A method according to claim 1 wherein said active agent is a lysosomal enzyme.

12-14. (canceled)

15. A method according to claim 11 wherein said active agent is Beta-glucocerebrosidase (β-glucocerebrosidase; GCase) or a derivative or analogue thereof.

16-19. (canceled)

20. A method according to claim 15 wherein said active agent is selected from the group consisting of alglucerase, imiglucerase, velaglucerase, or taliglucerase, and combinations thereof.

21-24. (canceled)

25. A method according to claim 1 wherein said active agent is a small molecule drug.

26. A method according to claim 1 wherein said Lewy body dementia is selected from dementia with Lewy bodies and Parkinson's disease dementia.

27-38. (canceled)

39. A method according to claim 1 wherein said therapeutic payload is administered according to a regimen selected from the group consisting of at least about once per day, or at least about every other day, or at least about two times per week, or at least about 1 time per week, or at least about 1 time every two weeks, or at least about 1 time per month.

40-42. (canceled)

43. A conjugate comprising p97 or a fragment thereof that is conjugated to an active agent suitable for treating Lewy body dementia to form aconjugate-p97-actrve agent conjugate wherein the p97 fragment comprises, consists essentially of, or consists of DSSHAFTLDELR (SEQ ID NO: 13), or a sequence having at least about 70% or more homology thereto.

44. A conjugate according to claim 43 wherein the p97 fragment has one or more terminal cysteines and/or tyrosines.

45-50. (canceled)

51. A conjugate comprising p97 or a fragment thereof that is conjugated to an active agent suitable for treating Lewy body dementia to form aconjugate-p97-actrve agent conjugate wherein the p97 fragment comprises, consists essentially of, or consists of DSSYSFTLDELR (SEQ ID NO: 19), or a sequence having at least about 70% or more homology thereto.

52. A conjugate according to claim 51 wherein the p97 fragment has one or more terminal cysteines and/or tyrosines.

53-58. (canceled)

59. A conjugate according to claim 43 wherein said active agent is a lysosomal enzyme.

60-62. (canceled)

63. A conjugate according to claim 59 wherein said active agent is Beta-glucocerebrosidase (β-glucocerebrosidase; GCase) or a derivative or analogue thereof.

64-67. (canceled)

68. A conjugate according to claim 59 wherein said active agent is selected from the group consisting of alglucerase, imiglucerase velaglucerase, or taliglucerase, and combinations thereof.

69-72. (canceled)

73. A conjugate according to claim 43 wherein said active agent is a small molecule drug.

74. A conjugate according to claim 51 wherein said active agent is a lysosomal enzyme.

75. A conjugate according to claim 74 wherein said active agent is Beta-glucocerebrosidase (β-glucocerebrosidase; GCase) or a derivative or analogue thereof.

76. A conjugate according to claim 75 wherein said active agent is selected from the group consisting of alglucerase, imiglucerase, velaglucerase, or taliglucerase, and combinations thereof.

77. A conjugate according to claim 51 wherein said active agent is a small molecule drug.
